(19) United States
(12) Patent Application Publication
(10) Pub. No.: US 2010/0280005 A1
(43) Pub. Date: Nov. 4, 2010

(54) Renin Inhibitors

(76) Inventors: Jonh J. Baldwin, Gwyneed Valley, PA (US); Salvacion Cacatian, Blue Bell, PA (US); David Claremon, Maple Glen, PA (US); Lawrence W. Dillard, Yardley, PA (US); Patrick T. Flaherty, Pittsburgh, PA (US); Bahman Ghavimi-Alagha, Wilmington, DE (US); John Gleason, King of Prussia, PA (US); Alexey V. Ishchenko, Somerville, MA (US); Brian Lawhorn, King of Prussia, PA (US); Gerard McGeehan, Garnet' Valley, PA (US); Jaclyn R. Patterson, King of Prussia, PA (US); Simon F. Semus, Collegeville, PA (US); Robert D. Simpson, Wilmington, DE (US); Suresh B. Singh, Kendall Park, NJ (US); Colin Tice, Ambler, PA (US); Tritin Tran, King of Prussia, PA (US); Zhenrong Xu, Horsham, PA (US); Jing Yuan, Lansdale, PA (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/450,620

(22) PCT Filed: Apr. 4, 2008
(86) PCT No.: PCT/US2008/059389

§ 371 Date: Jul. 14, 2010

Related U.S. Application Data
(60) Provisional application No. 60/910,223, filed on Apr. 5, 2007.

(51) Int. Cl. A61K 31/445; C07D 211/60; C07D 401/02; C07D 413/02; A61K 31/454; A61K 31/4545; A61K 31/4709; A61K 31/496; A61K 31/5377; A61K 31/5415; A61K 31/55; A61K 31/58; A61P 9/12

(52) U.S. Cl. 514/212.07; 546/245; 546/208; 546/189; 546/146; 544/365; 544/130; 514/330; 514/326; 514/316; 514/307; 514/253.13; 514/235.5; 514/223.5; 514/171

(57) ABSTRACT

Disclosed are compounds of Formula I, wherein the R, R¹, R^{2,} R³, X, Y, A, Q, E, and G are defined herein. These compounds bind to aspartic proteases to inhibit their activity and are useful in the treatment or amelioration of diseases associated with aspartic protease activity. Also disclosed are methods of use of the compounds of Formula I for ameliorating or treating aspartic protease related disorders in a subject in need thereof.

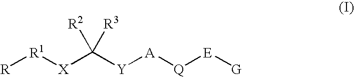

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin I, the product of renin catalyzed cleavage of angioteninogen are present. Elevated levels of β-amyloid, the product of BACE activity on amyloid precursor protein, are widely believed to be responsible for the amyloid plaques present in the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001,56,21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

Compounds have now been found which are orally active and bind to aspartic proteases to inhibit their activity. They are useful in the treatment or amelioration of diseases associated with aspartic protease activity.

In one embodiment the present invention is directed to compounds represented by Formula I:

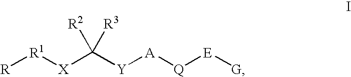

or an enantiomer, diastereomer or salt thereof, wherein:

R is:

a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁ -C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;

b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulflnyl, (C₃-C₆)cycloalkanesulfinyl, (C₄-C₇)cycloalkylalkanesulflnyl, halo(C₁-C₆)alkane-sulflnyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

c) a divalent radical selected from —(CH₂)₃—, —(CH₂)₄—, —(CH₂)₅— and —(CH₂)₆—, which is attached to R¹ to form a fused or spiro-fused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁ -C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁ -C₆)alkylaminocarbonyl;

X and Y are each independently CH₂ or a single bond;

R² is a) —H; or b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, amino sulfonylamino(C₁-C₁₂)alkyl, amino sulfonylamino(C₁-C₁₂)alkoxy, amino sulfonylamino(C₁-C₁₂)alkylthio, amino sulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkane sulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and/or

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;

wherein the divalent sulfur atoms in R² are independently optionally oxidized to sulfoxide or sulfone and wherein the carbonyl groups are optionally independently changed to a thiocarbonyl groups;

R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that

i) R² and R³ are not both hydrogen; and

ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C_{1 2})alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl)C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl)C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and/or

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;

The divalent sulfur atoms in R³ are independently optionally oxidized to sulfoxide or sulfone and wherein the carbonyl groups in R³ are optionally independently changed to thiocarbonyl groups;

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups and oxo groups such that when there is substitution with one oxo group on a carbon atom it forms a carbonyl group and when there is substitution of one or two oxo groups on sulfur it forms sulfoxide or sulfone groups, respectively, where m is 1 to 3;

Q and Y are attached to carbon or nitrogen atoms in ring A in a 1,2 or 1,3, or 1,4 relationship;

Q is a divalent radical:

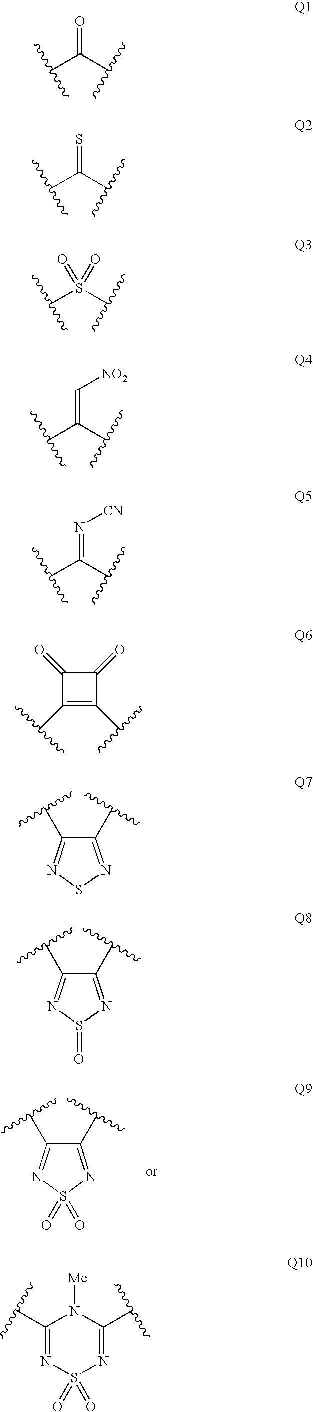

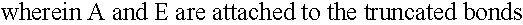

E is E¹ or —(C₁-C₃)alkyl-E¹; where E¹ is a 5-membered heteroaryl group containing 1-4 nitrogen atoms; an arylheterocyclyl group, wherein the heterocyclyl moiety contains 1-2 nitrogen atoms; or a saturated 4-, 5-, 6-, or 7-membered heterocyclic ring which is optionally bridged by (CH₂)ₙ via bonds to two members of the ring, wherein the ring is composed of carbon atoms and 1-3 heteroatoms selected from 1, 2, or 3 nitrogen atoms, 0 or 1 oxygen atoms, and 0 or 1 sulfur atoms; wherein E¹ is optionally substituted with one to three groups independently selected from halogen, hydroxy, (C₁-C₆)alkyl, halo(C₁-C₆)alkyl, hydroxy(C₁-C₆)alkyl, aryl, and oxo groups such that when there is substitution with one oxo group on a carbon atom it forms a carbonyl group and when there is substitution of one or two oxo groups on sulfur it forms sulfoxide or sulfone groups, respectively, where n is 1 to 3, and wherein G is attached to E¹ via a ring nitrogen atom, in a bonding arrangement illustrated as follows:

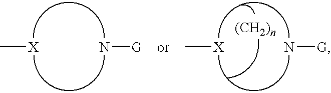

wherein X is a ring carbon atom or nitrogen atom bonded directly to Q or X is bonded to Q via the —(C₁-C₃)alkyl moiety of E;

and G is hydrogen, (C₁-C₆)alkyl, heterocyclyl, —(C₂-C₆)alkyl-OH, —(C₂-C₆)alkyl-NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino, where R^{4a} is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;

or E¹ is a 6-membered heteroaryl group containing 1-2 nitrogen atoms which is optionally substituted with one to two groups independently selected from halogen, (C₁-C₆)alkyl, halo(C₁-C₆)alkyl, hydroxy(C₁-C₆)alkyl, and aryl; and G is absent.

In another embodiment the present invention is directed to pharmaceutical compositions comprising a compound described herein or enantiomers, diastereomers, or salts thereof and a pharmaceutically acceptable carrier or excipient.

In another embodiment the present invention is directed to a method of inhibiting an aspartic protease in a subject in need thereof comprising administering to the subject a therapeutically effective amount of a compound described herein or an enantiomer, diastereomer, or salt thereof.

In another embodiment the present invention is directed to method for treating or ameliorating an aspartic protease mediated disorder in a subject in need thereof comprising administering to said subject a therapeutically effective amount of a compound described herein or an enantiomer, diastereomer, or salt thereof.

In another embodiment the present invention is directed to a method for treating or ameliorating a renin mediated disorder in a subject in need thereof comprising administering to the subject an effective amount of a compound described herein or an enantiomer, diastereomer, or salt thereof.

In another embodiment the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

A description of embodiments of the compounds of Formula I of the invention follows. It is understood that the invention encompasses all combinations of the substituent variables (i.e., R, R¹, R², R³, X, Y, A, Q, E and G) defined herein.

In one embodiment of this invention, R is: a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;

b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulflnyl, (C₃-C₆)cycloalkanesulfinyl, (C₄-C₇)cycloalkylalkanesulflnyl, halo(C₁-C₆)alkane-sulflnyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

c) a divalent radical selected from —(CH₂)₃—, —(CH₂)₄—, —(CH₂)₅— and —(CH₂)₆—, which is attached to R¹ to form a fused or spiro-fused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo.

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl.

X and Y are each independently CH₂ or a single bond.

R² is a) —H; or b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, amino sulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkanesulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and/or

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy.

The divalent sulfur atoms in R² are independently optionally oxidized to sulfoxide or sulfone and wherein the carbonyl groups are optionally independently changed to a thiocarbonyl groups;

R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that

i) R² and R³ are not both hydrogen; and

ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and/or

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy.

The divalent sulfur atoms in R³ are independently optionally oxidized to sulfoxide or sulfone and wherein the carbonyl groups in R³ are optionally independently changed to thiocarbonyl groups.

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups and oxo groups such that when there is substitution with one oxo group on a carbon atom it forms a carbonyl group and when there is substitution of one or two oxo groups on sulfur it forms sulfoxide or sulfone groups, respectively, where m is 1 to 3.

Q and Y are attached to carbon or nitrogen atoms in ring A in a 1,2 or 1,3, or 1,4 relationship.

Q is a divalent radical:

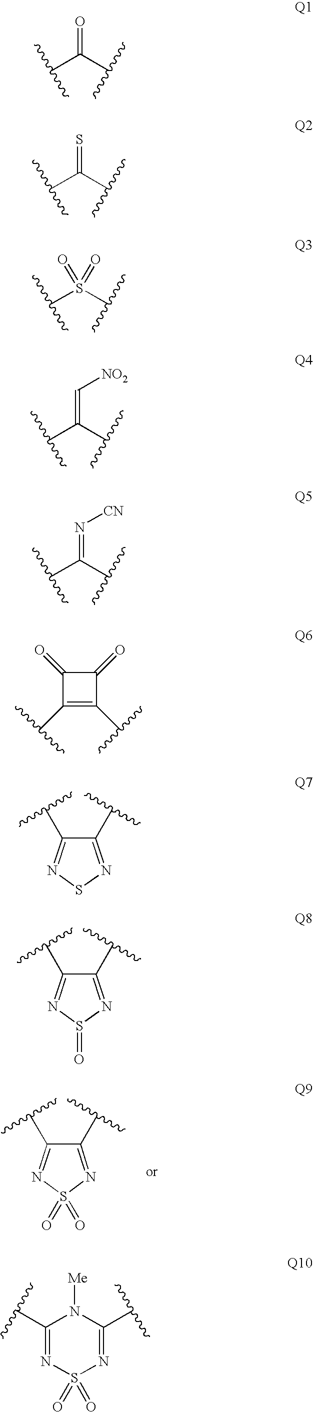

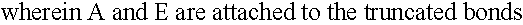

E is E¹ or —(C₁-C₃)alkyl-E¹; where E¹ is a saturated 4-, 5-, 6-, or 7-membered heterocyclic ring which is optionally bridged by (CH₂)ₙ via bonds to two members of said ring, wherein said ring is composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively, where n is 1 to 3, and wherein G is attached to ring E¹ via a ring nitrogen atom, in a bonding arrangement illustrated as follows:

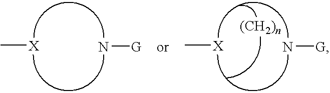

wherein X is a ring carbon atom or nitrogen atom bonded directly to Q or to the —(C₁-C₃)alkyl moiety of E.

G is hydrogen, (C₁-C₆)alkyl, heterocyclyl, —(C₂-C₆)alkyl-OH, —(C₂-C₆)alkyl-NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino, where R⁴a is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R⁴a, taken together with the nitrogen atom to which they are attached, form a 5-6 membered saturated heterocyclic ring composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively.

In another embodiment of this invention, R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from: fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or

(2) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulflnyl, (C₃-C₆)cycloalkanesulfinyl, (C₄-C₇)cycloalkylalkanesulfinyl, halo(C₁-C₆)alkane-sulflnyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO ₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

(3) R is a divalent radical selected from —(CH₂)₃—, —(CH₂)₄—, —(CH₂)₅— and —(CH₂)₆—, which is attached to R¹ to form a fused or spiro-fused ring system, and is optionally substituted with up to four substituents independently selected from: fluorine, hydroxy, (C₁-C₆)alkyl, halo(C₁-C₆)alkyl, (C₁-C₆)alkoxy and oxo.

In a particular embodiment of this invention, R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)-alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of: fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or

(2) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: fluorine, chlorine, cyano, (C₁-C₆)alkyl, (C₃-C₆)cycloalkyl, halo(C₁-C₆)alkyl, halo(C₃-C₆)cycloalkyl, (C₁-C₆)alkylthio, halo(C₁-C₆)alkylthio, (C₁-C₆)alkanesulfinyl, halo(C₁-C₆)alkanesulflnyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or

(3) R is a divalent radical selected from —(CH₂)₄— and —(CH₂)₅—, which is attached to R¹ to form a fused or spiro-fused ring system, and is optionally substituted with up to four substituents independently selected from: fluorine, hydroxy, (C₁-C₆)alkyl, halo(C₁-C₆)alkyl, (C₁-C₆)alkoxy and oxo.

In another particular embodiment, R is phenyl, naphthyl, monocyclic heteroaryl, bicyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or a divalent radical selected from —(CH₂)₄— and —(CH₂)₅—, which is attached to R¹ to form a fused or spiro-fused ring system.

In a further particular embodiment of this invention, R is phenyl, naphthyl, monocyclic heteroaryl, bicyclic heteroaryl, phenoxy, monocyclic heteroaryloxy, phenyl(C₁-C₃)alkoxy, phenyl(C₁-C₃)alkyl-, and monocyclic heteroaryl(C₁-C₃)alkoxy, each optionally substituted with up to 3 substituents independently selected from fluorine, chlorine, cyano, (C₁-C₃)alkyl, (C₃-C₆)cycloalkyl, halo(C₁-C₃)alkyl, (C₁-C₃)alkoxy, (C₁-C₃)alkylthio, and H₂NCO or with a divalent radical selected from —(CH₂)₄— and —(CH₂)₅—, which is attached to Rⁱ to form a fused ring system. In another particular embodiment of this invention, R is phenyl, naphthyl, monocyclic heteroaryl, bicyclic heteroaryl, phenoxy, monocyclic heteroaryloxy, phenyl(C₁-C₃)alkoxy, phenyl(C₁-C₃)alkyl-, and monocyclic heteroaryl(C₁-C₃)alkoxy, each optionally substituted with up to 3 substituents independently selected from fluorine, chlorine, (C₁-C₃)alkyl, (C₃-C₆)cycloalkyl, halo(C₁-C₃)alkyl, and (C₁-C₃)alkoxy. In specific embodiments of this invention, R is phenyl or phenoxy, each optionally substituted with 1 or 2 substituents independently selected from fluorine, chlorine and (C₁-C₃)alkyl; more specifically, R is 3-ethylphenyl or phenoxy.

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl, and di(C₁-C₆)alkylaminocarbonyl.

In a particular embodiment of this invention, R¹ is a phenyl, monocyclic heteroaryl, bicyclic heteroaryl, benzo-1,3-dioxole, or (C₃-C₇)cycloalkyl ring optionally substituted with up to four substituents independently selected from the group consisting of: fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl.

In another particular embodiment of this invention, R¹ is a phenyl or a monocyclic heteroaryl ring, optionally substituted with one to four substituents independently selected from: halogen, cyano, (C₁-C₃)alkyl, halo(C₁-C₃)alkyl, (C₁-C₃)alkoxy, halo(C₁-C₃)alkoxy, and H₂NCO. In specific embodiments of this invention, R¹ is a phenyl or a halogen-substituted phenyl group; more specifically R¹ is 6-fluorophenyl, 6-chlorophenyl or phenyl.

R² is (1) hydrogen or (2) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, amino sulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkane sulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, wherein (1) each optionally substituted by (a) 1 to 5 halogen atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone and wherein (3) a carbonyl group is optionally changed to a thiocarbonyl group.

In a particular embodiment of this invention, R² is, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino-(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)-alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxy-carbonylamino(C₁-C₅)alkoxy, (C₁-C₅)alkoxycarbonylamino(C₁-C₅)alkylthio, (C₁-C₅)alkoxy-carbonylamino(C₁-C₅)alkylamino, (C₁-C₅)alkylaminocarbonylamino(C₁-C₅)alkyl, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkylamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylamino-carboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)-alkanoylamino, wherein (1) each are optionally substituted by (a) 1 to 5 fluorine atoms and/or (b) 1 group selected from cyano, hydroxyl, (C₁-C₃)alkyl, (C₁-C₃)alkoxy, (C₃-C₄)cycloalkyl, (C₃-C₆)cycloalkoxy, halo(C₁-C₃)alkyl, halo(C₁-C₃)alkoxy, halo(C₃-C₄)cycloalkyl, and halo(C₃-C₆)cycloalkoxy and wherein (2) divalent sulfur atoms are optionally oxidized to sulfoxide or sulfone.

In another particular embodiment of this invention, R² is (C₁-C₈)alkoxy, (C₄-C₉)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅) (C₃-C₆)cycloalkoxy(C₁-C₅)alkyl, fluoro(C₁-C₅)alkoxy(C₁-C₅)alkyl, fluoro(C₃-C₆)cycloalkoxy(C₁-C₅)alkyl, (C₁-C₅)alkylthio(C₁-C₅)alkyl, (C₁-C₅) C₅)alkoxy, hydroxy(C₁-C₈)alkoxy, (C₃-C₆)cycloalkoxy(C₁-C₅)alkoxy, fluoro(C₁-C₅)alkoxy(C₁-C₅)alkoxy, fluoro(C₃-C₆)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, hydroxy-(C₁-C₃)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)-cycloalkanecarbonylamino(C₁-C₅)alkyl, (C₃-C₄)cycloalkanecarbonylamino(C₁-C₅)alkoxy, aminosulfonylamino(C₁-C₈)alkyl, aminosulfonylamino(C₁-C₈)alkoxy, (C₁-C₅)alkane-sulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylamino-carbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino.

In a further particular embodiment of this invention, R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, C₄)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy. In specific embodiments of this invention, R² is (C₁-C₃)alkoxy(C₁-C₅)alkyl or (C₁-C₃)alkoxycarbonylamino(C₁-C₅)alkyl; more specifically R² is 3-(methoxycarbonylamino)propyl or 4-methoxybutyl.

R³ is H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, or phenylamino or heteroarylamino in which each phenylamino and heteroarylamino group is optionally substituted with 1 to 5 groups independently selected from the group consisting of: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulflnyl, (C₃-C₆)cycloalkanesulfinyl, (C₄-C₇)cycloalkylalkanesulflnyl, halo(C₁-C₆)alkane-sulflnyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl; provided that (i) R² and R³ are not both hydrogen and (ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, wherein (1) each optionally substituted by (a) 1 to 5 halogen atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone and wherein (3) a carbonyl group is optionally changed to a thiocarbonyl group.

In another particular embodiment of this invention, R³ is hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylamino-carbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from: fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl; provided that when R³ is hydroxyl, or optionally substituted phenylamino or heteroarylamino, R² is not (C₁-C₁₀)alkoxy, (C₁-C₁₀)alkylthio, (C₁-C₁₀)alkylamino, (C₁-C₅)alkylthio(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, aminocarbonylamino(C₁-C₁₀)alkoxy, aminocarbonylamino(C₁-C₁₀)alkylthio, aminocarbonyl-amino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkylamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, wherein (1) each are optionally substituted by (a) 1 to 5 fluorine atoms and (b) by 1 group selected from cyano, hydroxyl, (C₁-C₃)alkyl, (C₁-C₃)alkoxy, (C₃-C₄)cycloalkyl, (C₃-C₆)cycloalkoxy, halo(C₁-C₃)alkyl, halo(C₁-C₃)alkoxy, halo(C₃-C₄)cycloalkyl, and halo(C₃-C₆)cycloalkoxy and wherein (2) divalent sulfur atoms are optionally oxidized to sulfoxide or sulfone.

In a further particular embodiment of this invention, R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy. In other embodiments of this invention, R³ is hydrogen or hydroxyl provided that when R³ is hydroxyl, R² is not 3-methoxypropoxy, 2-(acetylamino)ethoxy, or 2-(methoxycarbonylamino)ethoxy. In specific embodiments of this invention, R³ is hydroxyl.

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring atoms being substituted with the appropriate number of hydrogen atoms, said ring being optionally substituted with up to four independently selected halogen atoms, (C₁-C₆)alkyl groups, halo(C₁-C₆)alkyl groups and oxo groups such that when there is substitution with one oxo group on a carbon atom it forms a carbonyl group and when there is substitution of one or two oxo groups on sulfur it forms sulfoxide or sulfone groups, respectively; and, wherein m is 1 to 3. In one embodiment of the compounds of this invention, Ring A is a piperidine (piperidinyl) ring, a morpholine (morpholinyl) ring or a benzene (phenyl) ring. In another embodiment of the compounds of this invention, Ring A is a piperidinyl ring or a morpholinyl ring. In specific embodiments of this invention, Ring A is a piperidinyl ring.

Q and Y are attached to carbon or nitrogen atoms in ring A in a 1,2 or 1,3, or 1,4 relationship. In specific embodiments of this invention, Q and Y are attached to carbon or nitrogen atoms in ring A in a 1,3 relationship. X and Y are each independently CH₂ or a single bond. In the specific embodiments of this invention, X and Y are each a single bond.

In one embodiment of this invention, Q is a divalent radical:

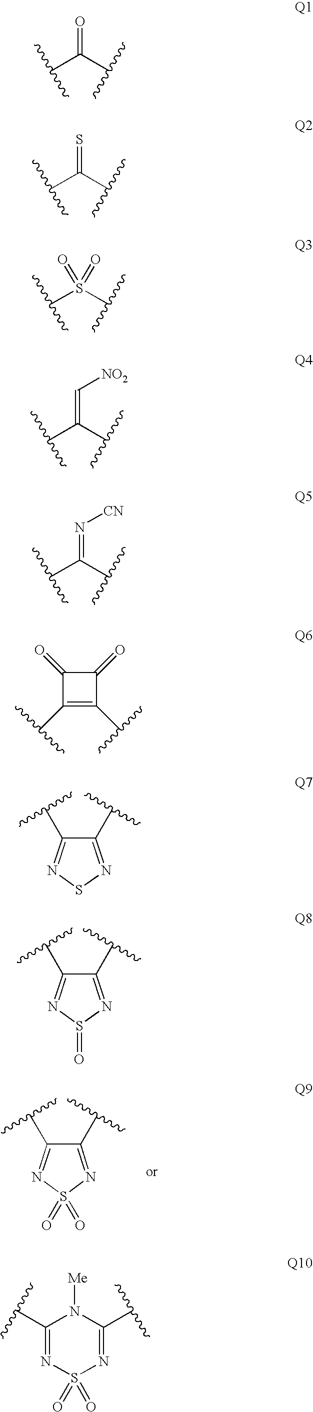

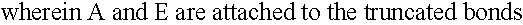

In another embodiment of this invention, Q is a divalent radical selected from Q1, Q2, Q3, Q4, Q5, Q6, and Q7. In a further embodiment of this invention, Q is Q1, Q2, Q3 or Q6. In specific embodiments of this invention, Q is Q1.

In a particular embodiment of this invention, E is E¹ or —(C₁-C₂)alkyl-E¹; where E¹ is a saturated 5- or 6-membered heterocyclic ring, wherein said ring is composed of carbon atoms and 1 or 2 heteroatoms selected from 1 or 2 nitrogen atoms, 0 or 1 oxygen atoms, and 0 or 1 sulfur atoms, said ring being optionally substituted with 1-2 groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively, wherein G is attached to ring E¹ via a ring nitrogen atom:

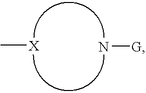

wherein X is a ring carbon atom or nitrogen atom bonded directly to Q or to the —(C₁-C₂)alkyl moiety of E. Accordingly, the bonding arrangement of groups -Q-E-G may be illustrated as follows:

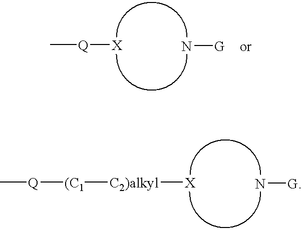

In a further embodiment E is E¹ or —(C₁-C₂)alkyl-E¹; where E¹ is a 5-membered heteroaryl group containing 1-4 nitrogen atoms or a 10-membered arylheterocyclyl group, wherein the heterocyclyl moiety contains 1-2 nitrogen atoms, wherein E¹ is optionally substituted with 1-2 groups independently selected from halogen, hydroxy, (C₁-C₆)alkyl, halo(C₁-C₆)alkyl, hydroxy(C₁-C₆)alkyl, aryl, and oxo groups such that when there is substitution with one oxo group on a carbon atom it forms a carbonyl group and when there is substitution of one or two oxo groups on sulfur it forms sulfoxide or sulfone groups, respectively, or E¹ is a 6-membered heteroaryl group containing 1-2 nitrogen atoms which is optionally substituted with one to two groups independently selected from halogen, (C₁-C₆)alkyl, halo(C₁-C₆)alkyl, hydroxy(C₁-C₆)alkyl, and aryl; and G is absent; and wherein the bonding or Q-E-G is described as above,

In another embodiment of this invention, E is E¹ or —(C₁-C₂)alkyl-E¹; where E¹ is a saturated 5- or 6-membered heterocyclic ring, wherein said ring is composed of carbon atoms and 1 or 2 nitrogen atoms, said ring being optionally substituted with one group selected from hydroxy, (C₁-C₄)alkyl, halo(C₁-C₄)alkyl, and hydroxy(C₁-C₄)alkyl.

In a further embodiment, E is E¹ or —(C₁-C₂)alkyl-E¹; where E¹ is a 5-membered heteroaryl group containing 1-4 nitrogen atoms; a 10-membered arylheterocyclyl group, wherein the heterocyclyl moiety contains one nitrogen atom;

or a saturated 5- or 6-membered heterocyclic ring, wherein said ring is composed of carbon atoms and 1 or 2 heteroatoms selected from 1 or 2 nitrogen atoms and 0 or 1 oxygen atoms, wherein E¹ is optionally substituted with 1-2 groups independently selected from hydroxy, (C₁-C₄)alkyl, halo(C₁-C₄)alkyl, hydroxy(C₁-C₄)alkyl, aryl, and oxo groups; or E¹ is a 6-membered heteroaryl group containing one nitrogen atom which is optionally substituted with one group selected from (C₁-C₄)alkyl, halo(C₁-C₄)alkyl, and hydroxy(C₁-C₄)alkyl, and G is absent.

In specific embodiments of this invention, E is E¹ or —(C₁-C₂)alkyl-E¹; where E¹ is selected from the group consisting of piperidinyl, piperazinyl, and pyrrolidinyl, said group being optionally substituted with a hydroxy, (C₁-C₃)alkyl or halo(C₁-C₃)alkyl group. In other specific embodiments, E¹ is selected from the group consisting of morpholinyl, 2,4-dioxo(1H, 3H)dihydropyrimidinyl (alternatively, 2,6-dioxohexahydropyrimidinyl), tetrazolyl, imidazolyl, pyridinyl, and tetrahydroisoquinolinyl, said group being optionally substituted with a hydroxy, phenyl, (C₁-C₃)alkyl, or halo(C₁-C₃)alkyl group. In more specific embodiments of this invention, E is piperidin-2-yl, piperidin-3-yl, piperidin-4-yl, piperazin-1-yl, pyrrolidin-2-nyl, pyrrolidine-3-yl, 4-hydroxy-pyrrolidin-2-yl-, piperidin-3-yl-methyl-, piperidin-4-yl-methyl-, piperazin-1-yl-methyl-, pyrrolidine-2-yl-methyl-, piperidin-2-yl-ethyl-, piperidin-4-yl-ethyl-,4-phenyl-piperidin-4-yl, morpholin-2-yl, morpholin-2-yl-methyl, 2,4-dioxo-dihydro(1H, 3H) pyrimidin-6-yl (alternatively, 2,6-dioxohexahydropyrimidin-4-y1), 1H-tetrazol-5-yl-methyl, 1H-imidazol-4-yl-methyl, pyridine-4-yl, pyridine-4-yl-methyl, 1,2,3,4-tetrahydroisoquinolin-6-yl, or 1,2,3,4-tetrahydroisoquinolin-7-yl. In other specific embodiments of this invention, E is piperidin-2-yl, piperidin-3-yl, piperidin-4-yl, piperazin-1-yl, pyrrolidin-2-nyl, pyrrolidine-3-yl, 4-hydroxy-pyrrolidin-2-yl-, piperidin-3-yl-methyl-, piperidin-4-yl-methyl-, piperazin-1-yl-methyl-, pyrrolidine-2-yl-methyl-, piperidin-2-yl-ethyl-, piperidin-4-yl-ethyl-,4-phenyl-piperidin-4-yl, morpholin-2-yl, morpholin-2-yl-methyl, 1H-tetrazol-5-yl-methyl, 1H-imidazol-4-yl-methyl, pyridine-4-yl, pyridine-4-yl-methyl, 1,2,3,4-tetrahydroisoquinolin-6-yl, or 1,2,3,4-tetrahydroisoquinolin-7-yl.

In one embodiment of the compounds of this invention, G is hydrogen, heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₇)cycloalkyl, or —(C₁-C₄)alkyl(C₄-C₇)heterocyclyl, wherein the (C₁-C₄)alkyl moiety of said —C(═O)(C₁-C₄)alkylaryl, —C(═O)(C₁-C₄)alkyl(C₄-C₇)heterocyclyl, —(C₁-C₄)alkyl(C₃-C₇)cycloalkyl and —(C₁-C₄)alkyl(C₄-C₇)heterocyclyl is optionally substituted by amino, hydroxy, or (C₁-C₃)alkylamino, where R⁴ is H or (C₁-C₃)alkyl and R^{4a} is selected from H, (C₁-C₃)alkyl, heterocyclyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 heteroatoms selected from 1, 2, or 3 nitrogen atoms and 0 or 1 oxygen atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group. In another embodiment of this invention, at least one of R⁴ and R^{4a} is H.

In one embodiment of the compounds of this invention, G is hydrogen, heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₇)cycloalkyl, or —(C₁-C₄)alkyl(C₄-C₇)heterocyclyl, wherein the (C₁-C₄)alkyl moiety of said —C(═O)(C₁-C₄)alkylaryl, —C(═O)(C₁-C₄)alkyl(C₄-C₇)heterocyclyl, —(C₁-C₄)alkyl(C₃-C₇)cycloalkyl and —(C₁-C₄)alkyl(C₄-C₇)heterocyclyl is optionally substituted by amino, hydroxy, or (C₁-C₃)alkylamino, where R⁴ is H or (C₁-C₃)alkyl and R^{4a} is selected from H, (C₁-C₃)alkyl, heterocyclyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 nitrogen atoms, specifically 2-3 nitrogen atoms, more specifically 2 nitrogen atoms; said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group. In another embodiment of this invention, at least one of R⁴ and R^{4a} is H.

In one embodiment of the compounds of this invention, G is hydrogen, (C₄-C₆)heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylphenyl, —C(═O)(C₁-C₄)alkyl(C₄-C₆)heterocyclyl, —(C₁-C₄)alkyl(C₃-C₆)cycloalkyl, or —(C₁-C₄)alkyl(C₄-C₆)heterocyclyl, wherein the (C₁-C₄)alkyl moiety of said —C(═O)(C₁-C₄)alkylphenyl, —C(═O)(C₁-C₄)alkyl(C₄-C₆)heterocyclyl, —(C₁-C₄)alkyl(C₃-C₆)cycloalkyl and —(C₁-C₄)alkyl(C₄-C₆)heterocyclyl is optionally substituted by amino, hydroxy, or (C₁-C₃)alkylamino, where R⁴ is H or (C₁-C₃)alkyl and R^{4a} is selected from H, (C₁-C₃)alkyl, (C₃-C₆)cycloalkyl(C₁-C₄)alkyl, and (C₄-C₆)heterocyclyl(C₁-C₄)alkyl.

In one embodiment of the compounds of this invention, G is hydrogen, (C₅-C₆)heterocyclyl, —(C₂-C₃)alkyl-OH, —C(═O)(C₁-C₂)alkyl-NR⁴R^{4a}, or —C(═O)(C₁-C₃)alkylphenyl, wherein the (C₁-C₃)alkyl moiety of said —C(═O)(C₁-C₃)alkylphenyl is substituted by amino or (C₁-C₃)alkylamino, where R⁴ is H or (C₁-C₃)alkyl and R^{4a} is H. In more specific embodiments of this invention, G is H, 2-hydroxyethyl, aminoacetyl-, piperidin-4-yl, or (3-phenyl, 2-amino)propanoyl- (or phenylalanyl).

An embodiment of the invention is a compound of Formula Ia:

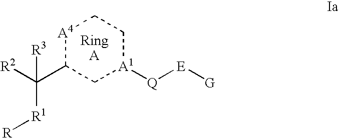

wherein R, R¹, R², R³, Q, E and G are as defined above for Formula I, and Ring A is a benzene ring (A¹ is C and A⁴ is CH and the bonds in ring A are aromatic bonds); or Ring A is a piperidine ring (A¹ is N, A⁴ is CH₂ and the bonds in ring A are single bonds); or Ring A is a morpholine ring (A¹ is N, A⁴ is O and the bonds in ring A are single bonds), or an enantiomer, diastereomer or salt thereof.

Another embodiment of the invention is a compound of Formula Ia with the stereochemical configuration shown in Formula Ib:

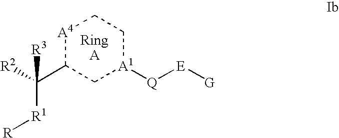

wherein R, R¹, R², R³, Ring A, A¹, A⁴, Q, E and G are as defined above, or an enantiomer, diastereomer or salt thereof. Specific and particular values for each variable in Formula Ib are as described above.

A further embodiment of invention is a compound of Formula Ic:

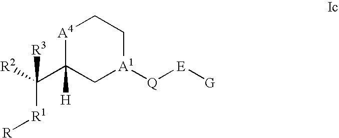

wherein R, R¹, R², R³, A, A¹, A⁴, Q, E and G are as defined above, or an enantiomer, diastereomer or salt thereof. Specific and particular values for each variable in Formula Ic are as described above.

One particular embodiment of the invention is a compound of Formula I, Ia, Ib, or Ic wherein:

E is E¹ or —(C₁-C₂)alkyl-E¹; where E¹ is a saturated 5- or 6-membered heterocyclic ring, wherein said ring is composed of carbon atoms and 1 or 2 nitrogen atoms, said ring being optionally substituted with one group selected from hydroxy, (C₁-C₄)alkyl, halo(C₁-C₄)alkyl, and hydroxy(C₁-C₄)alkyl; or

E¹ is a 5-membered heteroaryl group containing 1-4 nitrogen atoms; a 10-membered arylheterocyclyl group, wherein the heterocyclyl moiety contains one nitrogen atom, wherein E¹ is optionally substituted with 1-2 groups independently selected from hydroxy, (C₁-C₄)alkyl, halo(C₁-C₄)alkyl, hydroxy(C₁-C₄)alkyl, aryl, and oxo groups; and

G is hydrogen, (C₅-C₆)heterocyclyl, —(C₂-C₃)alkyl-OH, —C(═O)(C₁-C₂)alkyl-NR⁴R^{4a}, or —C(═O)(C₁-C₃)alkylphenyl, wherein the (C₁-C₃)alkyl moiety of said —C(═O)(C₁-C₃)alkylphenyl is substituted by amino or (C₁-C₃)alkylamino, where R⁴ is H or (C₁-C₃)alkyl and R^{4a} is H, or a salt thereof.

or E¹ is a 6-membered heteroaryl group containing one nitrogen atom which is optionally substituted with one group selected from (C₁-C₄)alkyl, halo(C₁-C₄)alkyl, and hydroxy(C₁-C₄)alkyl, and G is absent;

R is phenyl, naphthyl, monocyclic heteroaryl, bicyclic heteroaryl, phenoxy, monocyclic heteroaryloxy, phenyl(C₁-C₃)alkoxy, and monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio, and H₂NCO; or a divalent radical selected from —(CH₂)₄— and —(CH₂)₅—;

R¹ is a phenyl or a monocyclic heteroaryl ring, optionally substituted with up to four substituents independently selected from: halogen, cyano, (C₁-C₃)alkyl, halo(C₁-C₃)alkyl, (C₁-C₃)alkoxy, halo(C₁-C₃)alkoxy, and H₂NCO;

R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy; and

R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy;

or a salt thereof.

Further embodiments of this invention, comprise compounds of Formula I, Ia, Ib, or Ic wherein E, G, R, R¹, R² and R³ are as defined hereinabove, and A or Ring A is a piperidine ring or a morpholine ring, where for Formulas I, Ia, Ib and Ic: Ring A is a piperidine ring, where A¹ is N, A⁴ is CH₂ and the bonds in ring A are single bonds, or Ring A is a morpholine ring, where A¹ is N, A⁴ is O and the bonds in ring A are single bonds); and Q is Q1.

Another particular embodiment of the invention is a compound of Formula I, Ia or Ib wherein, R is 3-ethylphenyl or phenoxy; R¹ is 6-fluorophenyl, 6-chlorophenyl or phenyl; R² is 3-(methoxycarbonylamino)propyl or 4-methoxybutyl; R³ is hydroxyl; A or Ring A is a piperidine ring; Q is Q1; E is piperidin-2-yl, piperidin-3-yl, piperidin-4-yl, piperazin-1-yl, pyrrolidin-2-nyl, pyrrolidine-3-yl, 4-hydroxy-pyrrolidin-2-yl-, piperidin-3-ylmethyl-, piperidin-4-ylmethyl-, piperazin-1-ylmethyl-, pyrrolidine-2-ylmethyl-, piperidin-2-ylethyl-, piperidin-4-ylethyl-; G is H, 2-hydroxyethyl, aminoacetyl-, piperidin-4-yl, or (3-phenyl, 2-amino)propanoyl-; or an enantiomer, diastereomer or salt thereof

Other embodiments of the invention include compounds wherein:

R is phenyl, naphthyl, monocyclic heteroaryl, bicyclic heteroaryl, phenoxy, monocyclic heteroaryloxy, phenyl(C₁-C₃)alkoxy, and monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio, and H₂NCO; or a divalent radical selected from —(CH₂)₄— and —(CH₂)₅—;

R¹ is a phenyl or a monocyclic heteroaryl ring, optionally substituted with up to four substituents independently selected from: halogen, cyano, (C₁-C₃)alkyl, halo(C₁-C₃)alkyl, (C₁-C₃)alkoxy, halo(C₁-C₃)alkoxy, and H₂NCO;

R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy;

R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy;

A or Ring A is a piperidine ring or a morpholine ring; Q is Q1;

E is E¹ or —(C₁-C₂)alkyl-E¹; where E¹ is a saturated 5- or 6-membered heterocyclic ring, wherein said ring is composed of carbon atoms and 1 or 2 nitrogen atoms, said ring being optionally substituted with one group selected from hydroxy, (C₁-C₄)alkyl, halo(C₁-C₄)alkyl, and hydroxy(C₁-C₄)alkyl;

G is hydrogen, (C₅-C₆)heterocyclyl, —(C₂-C₃)alkyl-OH, —C(═O)(C₁-C₂)alkyl-NR⁴R^{4a}, or —C(═O)(C₁-C₃)alkylphenyl, wherein the (C₁-C₃)alkyl moiety of said —C(═O)(C₁-C₃)alkylphenyl is substituted by amino or (C₁-C₃)alkylamino, where R⁴ is H or (C₁-C₃)alkyl and R^{4a} is H;

or a salt thereof.

A further embodiment of this invention includes a compound of Formula I wherein R is 3-methylphenyl, 3-ethylphenyl, or phenoxy; R¹ is 6-fluorophenyl, 6-chlorophenyl or phenyl; R² is 3-(N-acetylamino)propyl, 3-(methoxycarbonylamino)propyl, or 4-methoxybutyl; R³ is hydroxyl; A or Ring A is a piperidine ring; Q is Q1; E is piperidin-2-yl, piperidin-3-yl, piperidin-4-yl, 4-phenyl-piperidin-4-yl, piperazin-1-yl, morpholin-2-yl, 2,4-dioxo-dihydro(1H, 3H) pyrimidin-6-yl (alternatively, 2,6-dioxohexahydropyrimidin-4-yl), pyrrolidin-2-yl, pyrrolidin-3-yl, 4-hydroxy-pyrrolidin-2-yl, piperidin-3-ylmethyl-, piperidin-4-ylmethyl-, piperazin-1-ylmethyl-, morpholin-2-ylmethyl-, pyrrolidine-2-ylmethyl-, piperidin-2-ylethyl-, piperidin-2-ylethyl-, piperidin-4-ylethyl-, 1H-tetrazol-5-ylmethyl-, 1H-imidazol-4-ylmethyl-, pyridin-4-yl, pyridin-4-ylmethyl-, tetrahydroisoquinolin-6-yl, and tetrahydroisoquinolin-7-yl; G is H, 2-hydroxyethyl-, aminoacetyl-, piperidin-4-yl, or (2-amino-3-phenyl)propanoyl-; or a salt thereof.

It will be appreciated by those skilled in the art, that the compounds of this invention contain 1, 2 or more chiral centers and may exist in different enantiomeric and/or diastereomeric forms. The following compounds are recited without reference to the relative or absolute configuration of any of the chiral centers present therein, but such recitation is intended to encompass each enantiomeric and/or diastereomeric form of these compounds and all mixtures thereof, such as enantiomerically and/or diastereomerically enriched mixtures and racemic mixtures. The following are compounds of the invention:

| Cpd. No. | Name |
|---|---|
| I-1 | Methyl (4-(3′-ethyl-6-fluoro-2-biphenylyl)-4-hydroxy-4-{1-[4-hydroxy-L-prolyl]- |
| | 3-piperidinyl}butyl)carbamate |
| I-2 | Methyl (4-(3′-ethyl-6-fluoro-2-biphenylyl)-4-hydroxy-4-{1-[3- |
| | pyrrolidinylcarbonyl]-3-piperidinyl}butyl)carbamate |
| I-3 | (3-(1-Hydroxy-5-methoxy-1-(2-phenoxyphenyl)pentyl)piperidin-1- |
| | yl)(piperidin-4-yl)methanone |
| I-4 | Methyl (4-(3′-ethyl-6-fluoro-2-biphenylyl)-4-hydroxy-4-{1-[2- |
| | pyrrolidinylacetyl]-3-piperidinyl}butyl)carbamate |
| I-5 | Methyl {4-(3′-ethyl-6-fluoro-2-biphenylyl)-4-hydroxy-4-[1-(2- |
| | piperidinylcarbonyl)-3-piperidinyl]butyl}carbamate |
| I-6 | Methyl (4-(3′-ethyl-6-fluoro-2-biphenylyl)-4-hydroxy-4-{1-[3- |
| | piperidinylcarbonyl]-3-piperidinyl}butyl)carbamate |
| I-7 | Methyl {4-(3′-ethyl-6-fluoro-2-biphenylyl)-4-hydroxy-4-[1-(4- |
| | piperidinylcarbonyl)-3-piperidinyl]butyl}carbamate |
| I-8 | Methyl {4-(3′-ethyl-6-fluoro-2-biphenylyl)-4-hydroxy-4-[1-(4- |
| | piperidinylacetyl)-3-piperidinyl]butyl}carbamate |
| I-9 | Methyl {4-(3′-ethyl-6-fluoro-2-biphenylyl)-4-hydroxy-4-[1-(3- |
| | piperidinylacetyl)-3-piperidinyl]butyl}carbamate |
| I-10 | Methyl (4-(3′-ethyl-6-fluoro-2-biphenylyl)-4-hydroxy-4-{1-[3-(4- |
| | piperidinyl)propanoyl]-3-piperidinyl}butyl)carbamate |
| I-11 | methyl (4-(3′-ethyl-6-fluoro-2-biphenylyl)-4-hydroxy-4-{1-[3-(2- |
| | piperidinyl)propanoyl]-3-piperidinyl}butyl)carbamate |
| I-12 | methyl (4-(3′-ethyl-6-fluoro-2-biphenylyl)-4-{1-[(1-glycyl-4- |
| | piperidinyl)carbonyl]-3-piperidinyl}-4-hydroxybutyl)carbamate |
| I-13 | methyl (4-(3′-ethyl-6-fluoro-2-biphenylyl)-4-hydroxy-4-{1-[1-(4- |
| | piperidinyl)-L-prolyl]-3-piperidinyl}butyl)carbamate |
| I-14 | methyl {4-(3′-ethyl-6-fluoro-2-biphenylyl)-4-hydroxy-4-[1-(L- |
| | phenylalanyl-L-prolyl)-3-piperidinyl]butyl}carbamate |
| I-15 | methyl [4-(3′-ethyl-6-fluoro-2-biphenylyl)-4-hydroxy-4-(1-{[4-(2- |
| | hydroxyethyl)-1-piperazinyl]acetyl}-3-piperidinyl)butyl]carbamate |
| I-16 | methyl {4-(6-chloro-3′-ethyl-2-biphenylyl)-4-hydroxy-4-[1-(1- |
| | piperazinylcarbonyl)-3-piperidinyl]butyl}carbamate |
| I-17 | methyl [4-(6-chloro-3′-methyl-2-biphenylyl)-4-(1-{[2,6-dioxohexahydro- |
| | 4-pyrimidinyl]carbonyl}-3-piperidinyl)-4-hydroxybutyl]carbamate |
| I-18 | methyl [4-(6-chloro-3′-methyl-2-biphenylyl)-4-(1-{[2,6-dioxohexahydro- |
| | 4-pyrimidinyl]carbonyl}-3-piperidinyl)-4-hydroxybutyl]carbamate |
| I-19 | methyl (4-(3′-ethyl-6-fluoro-2-biphenylyl)-4-hydroxy-4-{1-[(4-phenyl-4- |
| | piperidinyl)carbonyl]-3-piperidinyl}butyl)carbamate |
| I-20 | methyl {4-(3′-ethyl-6-fluoro-2-biphenylyl)-4-hydroxy-4-[1-(2- |
| | morpholinylcarbonyl)-3-piperidinyl]butyl}carbamate |
| I-21 | methyl {4-(3′-ethyl-6-fluoro-2-biphenylyl)-4-hydroxy-4-[1-(2- |
| | morpholinylacetyl)-3-piperidinyl]butyl}carbamate |
| I-22 | 1-(6-chloro-3′-ethyl-2-biphenylyl)-5-(methyloxy)-1-[1-(1H-tetrazol-5- |
| | ylacetyl)-3-piperidinyl]-1-pentanol |
| I-23 | methyl {4-(6-chloro-3′-ethyl-2-biphenylyl)-4-hydroxy-4-[1-(1H-tetrazol- |
| | 5-ylacetyl)-3-piperidinyl]butyl}carbamate |
| I-24 | N-{4-(6-chloro-3′-methyl-2-biphenylyl)-4-hydroxy-4-[1-(1H-tetrazol-5- |
| | ylacetyl)-3-piperidinyl]butyl}acetamide |
| I-25 | N-{4-(6-chloro-3′-methyl-2-biphenylyl)-4-hydroxy-4-[1-(1H-imidazol-4- |
| | ylacetyl)-3-piperidinyl]butyl}acetamide |
| I-26 | methyl {4-(3′-ethyl-6-fluoro-2-biphenylyl)-4-hydroxy-4-[1-(4- |
| | pyridinylcarbonyl)-3-piperidinyl]butyl}carbamate |
| I-27 | methyl {4-(3′-ethyl-6-fluoro-2-biphenylyl)-4-hydroxy-4-[1-(4- |
| | pyridinylacetyl)-3-piperidinyl]butyl}carbamate |
| I-28 | methyl (4-(6-chloro-3′-ethyl-2-biphenylyl)-4-hydroxy-4-{1-[(4- |
| | piperidinylamino)carbonyl]-3-piperidinyl}butyl)carbamate |
| I-29 | methyl [4-(6-chloro-3′-ethyl-2-biphenylyl)-4-hydroxy-4-(1-{[(4- |
| | piperidinylmethyl)amino]carbonyl}-3-piperidinyl)butyl]carbamate |
| I-30 | methyl {4-(6-chloro-3′-ethyl-2-biphenylyl)-4-hydroxy-4-[1-(1,2,3,4- |
| | tetrahydro-6-isoquinolinylcarbonyl)-3-piperidinyl]butyl}carbamate |
| I-31 | methyl {4-(6-chloro-3′-ethyl-2-biphenylyl)-4-hydroxy-4-[1-(1,2,3,4- |
| | tetrahydro-7-isoquinolinylcarbonyl)-3-piperidinyl]butyl}carbamate |

or a diastereomer, enantiomer or salt thereof.

It will also be appreciated by those skilled in the art that each enantiomer and diastereomer of the compounds of this invention will likely demonstrate a different level of effectiveness of inhibiting the action of aspartic proteases, particularly renin. It will be further appreciated that for the most active compounds, all enantiomers and/or diastereomers may demonstrate some level of activity, but that for compounds with lower activity, certain enantiomers and/or diastereomers may demonstrate such low levels of activity as to be considered inactive. It is understood that the following represent the preferred relative and absolute configuration of the compounds of the invention. It will be appreciated that each of the different enantiomeric and/or diastereomeric forms of the compounds of this invention, including the stereoisomeric forms depicted below, may be separately obtained using conventional procedures (e.g. stereospecific synthesis or resolution via chiral chromatography, crystallization, etc.).

| Cpd. | | |
|---|---|---|
| No. | Structure | Name |
| I-la | 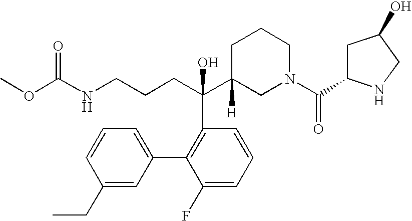 | methyl ((4S)-4-(3′-ethyl-6-fluoro-2- biphenylyl)-4-hydroxy-4-{(3R)-1-[(4R)-4- hydroxy-L-prolyl]-3- piperidinyl}butyl)carbamate |
| I-2a | 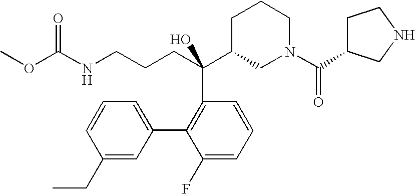 | methyl ((4S)-4-(3′-ethyl-6-fluoro-2- biphenylyl)-4-hydroxy-4-{(3R)-1-[(3R)-3- pyrrolidinylcarbonyl]-3- piperidinyl}butyl)carbamate |
| I-3a | 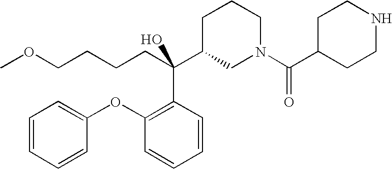 | ((R)-3-((S)-1-hydroxy-5-methoxy-1-(2- phenoxyphenyl)pentyl)piperidin-1- yl)(piperidin-4-yl)methanone |
| I-4a | 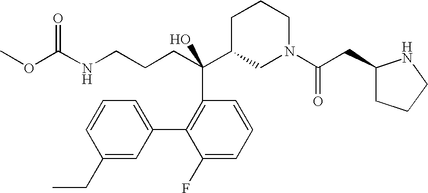 | methyl ((4S)-4-(3′-ethyl-6-fluoro-2- biphenylyl)-4-hydroxy-4-{(3R)-1-[(2S)-2- pyrrolidinylacetyl]-3- piperidinyl}butyl)carbamate |
| I-5a | 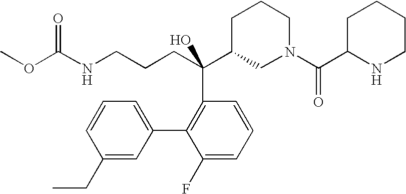 | methyl {(4S)-4-(3′-ethyl-6-fluoro-2- biphenylyl)-4-hydroxy-4-[(3R)-1-(2- piperidinylcarbonyl)-3- piperidinyl]butyl}carbamate |
| I-6a | 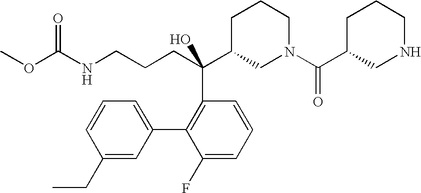 | methyl ((4S)-4-(3′-ethy1-6-fluoro-2- biphenylyl)-4-hydroxy-4-{(3R)-1-[(3R)-3- piperidinylcarbonyl]-3 piperidinyl}butyl)carbamate |
| I-7a | 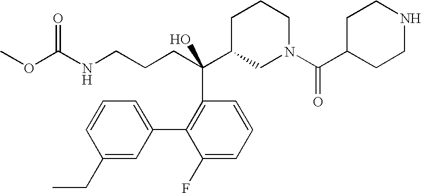 | methyl {(4S)-4-(3′-ethyl-6-fluoro-2- biphenylyl)-4-hydroxy-4-[(3R)-1-(4- piperidinylcarbony1)-3- piperidinyl]butyl}carbamate |
| I-8a | 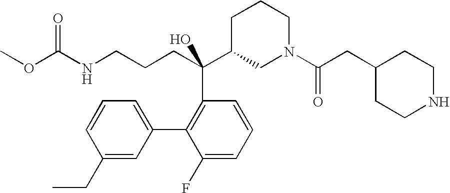 | methyl {(4S)-4-(3′-ethyl-6-fluoro-2- biphenylyl)-4-hydroxy-4-[(3R)-1-(4- piperidinylacetyl)-3- piperidinyl]butyl}carbamate |
| I-9a | 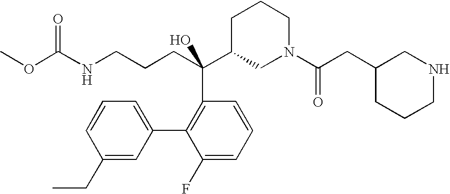 | methyl {(4S)-4-(3′-ethyl-6-fluoro-2- biphenylyl)-4-hydroxy-4-[(3R)-1-(3- piperidinylacetyl)-3- piperidinyl]butyl}carbamate |
| I-10a | 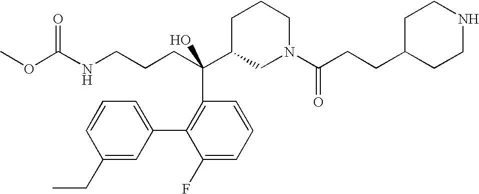 | methyl ((4S)-4-(3′-ethyl-6-fluoro-2- biphenylyl)-4-hydroxy-4-{(3R)-1-[3-(4- piperidinyl)propanoyl]-3- piperidinyl}butyl)carbamate |
| I-11a | 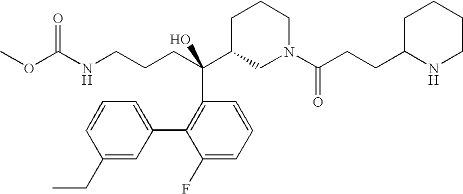 | methyl ((4S)-4-(3′-ethyl-6-fluoro-2- biphenylyl)-4-hydroxy-4-{(3R)-1-[3-(2- piperidinyl)propanoyl]3- piperidinyl}butyl)carbamate |
| I-12a | 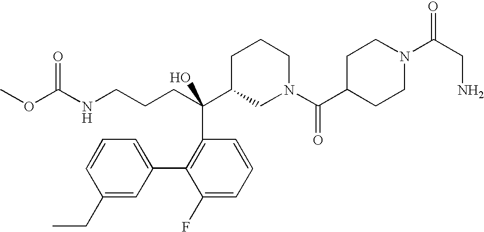 | methyl ((4S)-4-(3′-ethyl-6-fluoro-2- biphenylyl)-4-{(3R)-1-[(1-glycyl-4- piperidinyl)carbonyl]-3-piperidinyl}-4- hydroxybutyl)carbamate |
| I-13a | 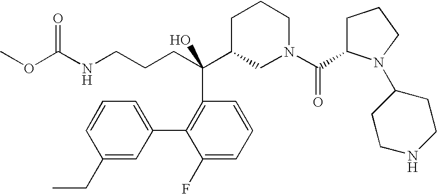 | methyl ((4S)-4-(3′-ethyl-6-fluoro-2- biphenylyl)-4-hydroxy-4-{(3R)-1-[1-(4- piperidinyl)-L-prolyl]-3- piperidinyl}butyl)carbamate |
| I-14a | 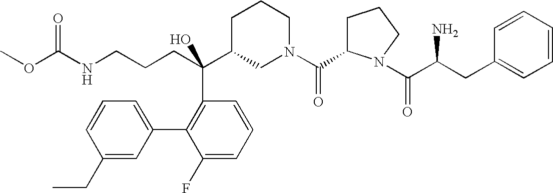 | methyl {(4S)-4-(3′-ethyl-6-fluoro-2- biphenylyl)-4-hydroxy-4[(3R)-1-(L- phenylalanyl-L-prolyl)-3- piperidinyl]butyl}carbamate |
| I-15a | 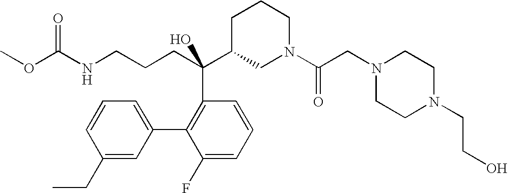 | methyl [(4S)-4-(3′-ethyl-6-fluoro-2- biphenylyl)-4-hydroxy-4((3R)-1-{[4-(2- hydroxyethyl)-1-piperazinyl]acetyl}-3- piperidinyl)butyl]carbamate |
| I-16a | 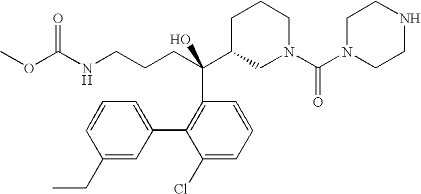 | methyl {(4S)-4-(6-chloro-3′-ethyl-2- biphenylyl)-4-hydroxy-4-[(3R)-1-(1- piperazinylcarbonyl)-3- piperidinyl]butyl}carbamate |
| I-17a | 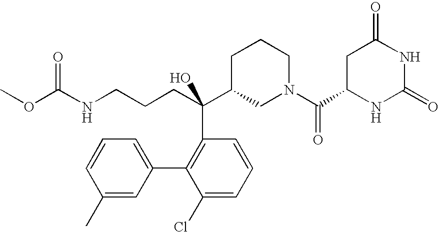 | methyl [(4S)-4-(6-chloro-3′-methyl-2- biphenylyl)-4-((3R)-1-{[(4S)-2,6- dioxohexahydro-4-pyrimidinyl]carbonyl}- 3-piperidinyl)-4-hydroxybutyl]carbamate |
| I-18a | 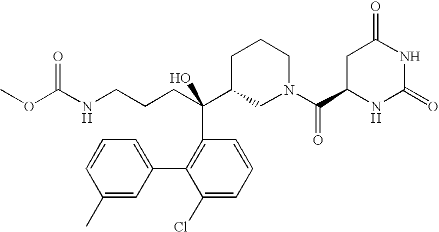 | methyl [(4S)-4-(6-chloro-3′-methyl-2- biphenylyl)-4-((3R)-1-{[(4R)-2,6- dioxohexahydro-4-pyrimidinyl]carbonyl}- 3-piperidinyl)-4-hydroxybutyl]carbamate |
| I-19a | 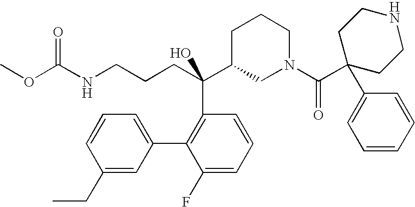 | methyl ((4S)-4-(3′-ethyl-6-fluoro-2- biphenyly1)-4-hydroxy-4-{(3R)-1-[(4- phenyl-4-piperidinyl)carbonyl]-3- piperidinyl}butyl)carbamate |
| I-20a | 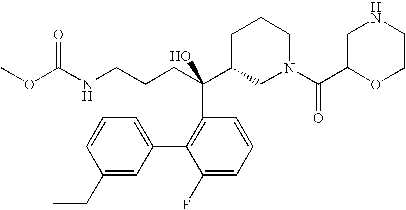 | methyl {(4S)-4-(3′-ethyl-6-fluoro-2- biphenylyl)-4-hydroxy-4-[(3R)-1-(2- morpholinylcarbonyl)-3- piperidinyl]butyl}carbamate |
| I-21a | 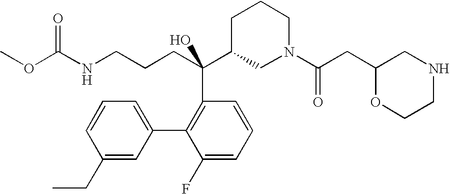 | methyl {(4S)-4-(3′-ethyl-6-fluoro-2- biphenylyl)-4-hydroxy-4-[(3R)-1-(2- morpholinylacetyl)-3- piperidinyl]butyl}carbamate |
| I-22a | 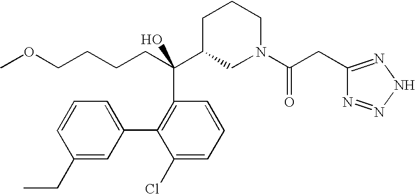 | (1S)-1-(6-chloro-3′-ethyl-2-biphenylyl)-5- (methyloxy)-1-[(3R)-1-(1H-tetrazol-5- ylacetyl)-3-piperidinyl]-1-pentanol |
| I-23a | 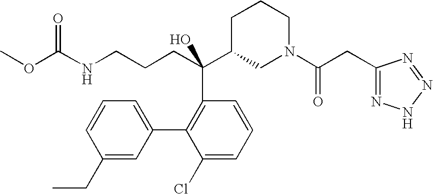 | methyl {(4S)-4-(6-chloro-3′-ethyl-2- biphenylyl)-4-hydroxy-4-[(3R)-1-(1H- tetrazol-5-ylacetyl)-3- piperidinyl]butyl}carbamate |
| I-24a | 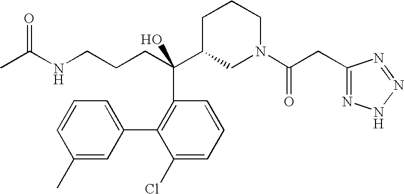 | N-{(4S)-4-(6-chloro-3′-methyl-2- biphenylyl)-4-hydroxy-4-[(3R)-1-(1H- tetrazol-5-ylacetyl)-3- piperidinyl]butyl}acetamide |
| I-25a | 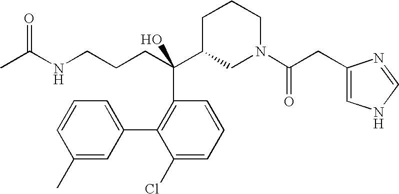 | N-{(4S)-4-(6-chloro-3′-methyl-2- biphenylyl)-4-hydroxy-4-[(3R)-1-(1H- imidazol-4-ylacetyl)-3- piperidinyl]butyl}acetamide |
| I-26a | 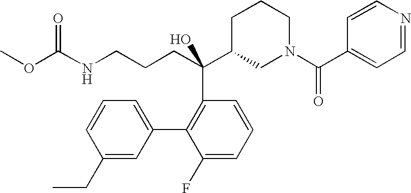 | methyl {(4S)-4-(3′-ethyl-6-fluoro-2- biphenylyl)-4-hydroxy-4-[(3R)-1-(4- pyridinylcarbonyl)-3- piperidinyl]butyl}carbamate |
| I-27a | 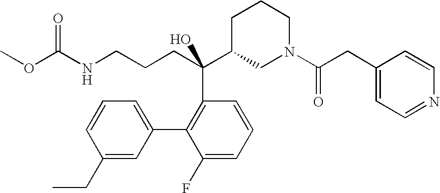 | methyl {(4S)-4-(3′-ethyl-6-fluoro-2- biphenylyl)-4-hydroxy-4-[(3R)-1-(4- pyridinylacetyl)-3- piperidinyl]butyl}carbamate |
| I-28a | 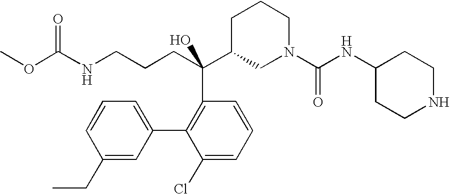 | methyl ((4S)-4-(6-chloro-3′-ethyl-2- biphenylyl)-4-hydroxy-4-{(3R)-1-[(4- piperidinylamino)carbonyl]-3- piperidinyl}butyl)carbamate |
| I-29a | 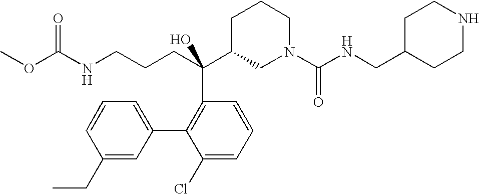 | methyl [(4S)-4-(6-chloro-3′-ethyl-2- biphenylyl)-4-hydroxy-4-((3R)-1-{[(4- piperidinylmethyl)amino]carbonyl}-3- piperidinyl)butyl]carbamate |
| I-30a | 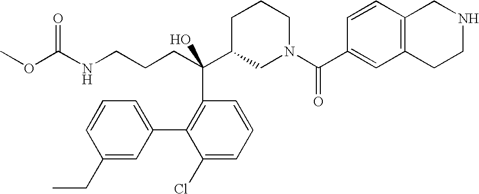 | methyl {(4S)-4-(6-chloro-3′-ethy1-2- biphenylyl)-4-hydroxy-4-[(3R)-1-(1,2,3,4- tetrahydro-6-isoquinolinylcarbonyl)-3- piperidinyl]butyl}carbamate |
| I-31a | 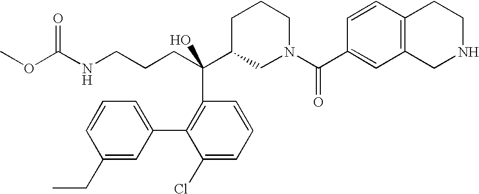 | methyl {(4S)-4-(6-chloro-3′-ethyl-2- biphenylyl)-4-hydroxy-4-[(3R)-1-(1,2,3,4- tetrahydro-7-isoquinolinylcarbonyl)-3- piperidinyl]butyl}carbamate |

or the salts thereof.

The following Compound Nos. represent preferred compounds of this invention: I-2a, I-10a, I-11a, I-12a, and I-22a, or a salt thereof. The following Compound Nos. represent the more preferred compounds of this invention: I-7a, I-8a, I-9a, I-16a, and I-21a, or a salt thereof.

The compounds of the invention (Compound # 1-31) exhibit 50% renin inhibition (as determined using the method of Example 9) at concentrations of from approximately 8000 nM to approximately 0.01 nM. Preferred compounds of the invention exhibit 50% inhibition at concentrations of from approximately 100 nM to approximately 0.01 nM. More preferred compounds of the invention exhibit 50% inhibition at concentrations of from approximately 5 nM to approximately 0.01 nM.

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-8 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

“Heterocyclyl” means a heteroaryl or a saturated heterocyclic ring group.

Saturated heterocyclic rings are 4-, 5-, 6-, and 7-membered heterocyclic rings containing 1 to 4 heteroatoms independently selected from N, 0, and S, and include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic and polycyclic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4,3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

“Alkoxy” means an alkyl radical attached through an oxygen linking atom. “(C₁-C₄)-alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Arylheterocyclyl” means a phenyl group fused to a partially saturated 5 or 6 membered heterocyclic ring containing 1-2 heteroatoms independently selected from N, S, and O, where at least 1 heteroatom is N. Examples of arylheterocyclyl groups include dihydrobenzoxazinyl, tetrahydroquinolinyl, tetrahydroisoquinolinyl, and tetrahydroquinoxalinyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

### Enantiomers, Diastereomers, and Salts

Certain compounds of Formula I may exist in various stereoisomeric or tautomeric forms. The invention encompasses all such forms of the compounds described herein, including an enantiomer or diastereomer thereof. The invention also encompasses active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The anionic salt form of a compound of the invention includes the acetate, bromide, camsylate, chloride, edisylate, fumarate, hydrobromide, hydrochloride, iodide, isethionate, lactate, mesylate, maleate, napsylate, salicylate, sulfate, and tosylate salts.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or non-aqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state.

Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R *” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by “ [symbol] ” which represents [symbol] or “—”.

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; arial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodeling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of Pamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I, comprise a pharmaceutically acceptable salt of a compound of Formula I or a prodrug or pharmaceutically active metabolite of such a compound or salt and one or more pharmaceutically acceptable carriers therefore.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of Formula I or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist. α-Blockers include doxazosin, prazosin, tamsulosin, and terazosin. β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts. Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts. A diuretic is, for example, a thiazide derivative selected from amiloride, chlorothiazide, hydrochlorothiazide, methylchlorothiazide, and chlorothalidon. ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril. Dual ACE/NEP inhibitors are, for example, omapatrilat, fasidotril, and fasidotrilat. Preferred ARBs include candesartan, eprosartan, irbesartan, losartan, olmesartan, tasosartan, telmisartan, and valsartan. Preferred aldosterone synthase inhibitors are anastrozole, fadrozole, and exemestane. Preferred aldosterone-receptor antagonists are spironolactone and eplerenone. A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of Formula I or composition thereof in a combination therapy with one or more additional agents for the treatment of AIDS including reverse transcriptase inhibitors, non-nucleoside reverse transcriptase inhibitors, other HIV protease inhibitors, HIV integrase inhibitors, attachment and fusion inhibitors, antisense drugs and immune stimulators. Preferred reverse transcriptase inhibitors are zidovudine, didanosine, zalcitabine, stavudine, lamivudine, abacavir, tenofovir, and emtricitabine. Preferred non-nucleoside reverse transcriptase inhibitors are nevirapine, delaviridine, and efavirenz. Preferred HIV protease inhibitors are saquinavir, ritonavir, indinavir, nelfinavir, amprenavir, lopinavir, atazanavir, and fosamprenavir. Preferred HIV integrase inhibitors are L-870,810 and S-1360. A preferred attachment and fusion inhibitor is enfuvirtide.

An embodiment of the invention includes administering β-secretase inhibiting compound of Formula I or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of Formula I or composition thereof in a combination therapy with one or more additional agents for the treatment of malaria including artemisinin, chloroquine, halofantrine, hydroxychloroquine, mefloquine, primaquine, pyrimethamine, quinine, sulfadoxine

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally).

The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

In the discussion below R, R¹, R², R³, X, Y, A, Q, E, and G are defined as described above for compounds of Formula I. In cases where the synthetic intermediates and final products of Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). In the discussion below all intermediates are assumed to be protected when necessary and protection/deprotection are generally not described.

In the first process of the invention, a compound of Formula I, in which a nitrogen atom that is part of A is attached to Q, is prepared by reaction of an amine of Formula II and an intermediate of Formula III:

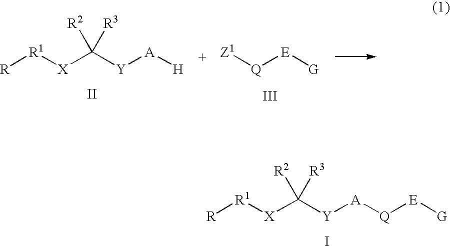

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula II wherein H is attached to a nitrogen atom that is part of A are prepared from intermediates of Formula IV:

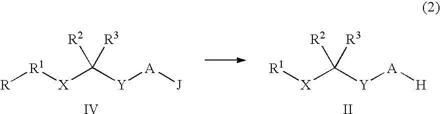

wherein J is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IV wherein R³═OH are prepared from ketone intermediates of formula V by addition of an organometallic reagent of formula VI, where M is for example Li, MgCl, MgBr, or MgI, to the carbonyl group of V:

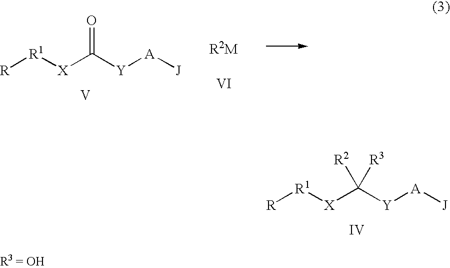

Intermediates of Formula IV wherein R³═H and R² is a group attached by an ether linkage are prepared from alcohol intermediates of formula VII by reaction with an alkylating agent under basic conditions or by reaction with an alcohol of formula R²OH under acidic conditions.

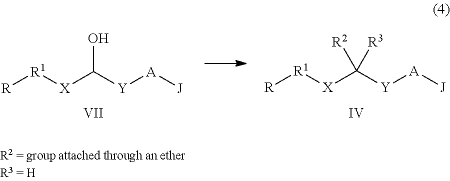

Alcohol intermediates of formula VII are prepared by reduction of ketone intermediates of formula V:

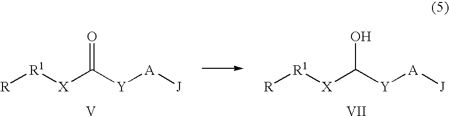

or by addition of an organometallic reagent of formula VIII, wherein M is, for example Li, MgCl, MgBr, or MgI, to an aldehyde of Formula IX:

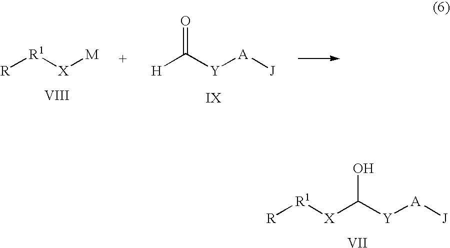

Ketone intermediates of formula V are prepared by the addition of an organometallic reagent of formula VIII, wherein M is Li, MgCl, MgBr, MgI, to a carboxylic acid derivative of formula X wherein Z² is an alkoxy, dialkylamino group, or an N-alkoxy-N-alkylamino group:

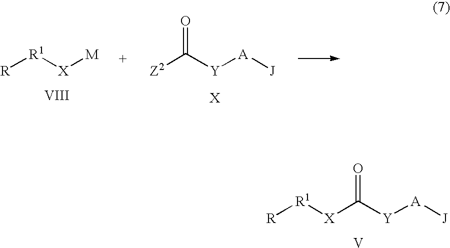

Intermediates of Formula III, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XV:

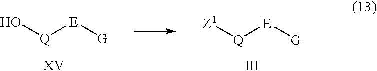

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula III are often prepared and used in situ without isolation.

In the second process of the invention, a compound of Formula I, in which a nitrogen atom that is part of E is attached to Q, is prepared by reaction of an intermediate of Formula XVIII and an amine of Formula XVI:

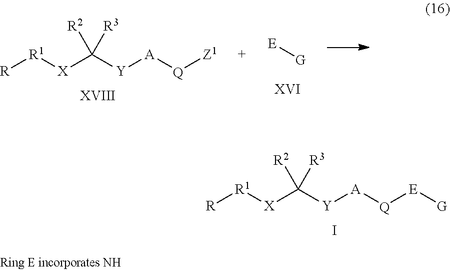

wherein Z¹ is as defined above.

Intermediates of Formula XVIII wherein Q is attached to a nitrogen atom of ring A and Q is Q1, Q4, Q5, Q6, Q8, Q9, or Q10 are prepared from amine intermediates of Formula II and intermediates of Formula XVII wherein Z¹ is halide, alkanesulfonate, haloalkanesulfonate, carboxylate, arylsulfonate, aryloxy, heteroaryloxy, azole, azolium salt, alkoxy, alkylthio, or arylthio:

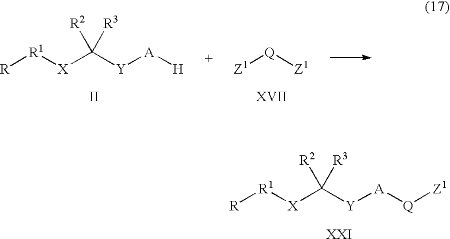

In the fifth process of the invention, a compound of Formula I, in which R is an alkoxy, cycloalkoxy, cycloalkylalkoxy or arylalkoxy group, is prepared by reaction of an alkylating agent of Formula XXII, in which Z³ is chloride, bromide, iodide, methanesulfonate, arenesulfonate or trifluoromethanesulfonate and Rc is an alkyl, cycloalkyl, cycloalkylalkyl or arylalkyl, with a hydroxy compound of Formula XXIII:

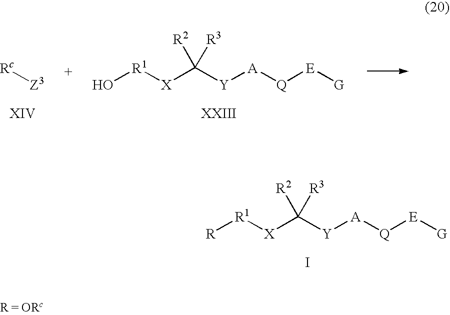

Intermediates of Formula XXIII are prepared by routes analogous to those shown for compounds of Formula I in reaction schemes 1 and 16.

In the first process of the invention, a compound of Formula Ia, in which A¹ is a nitrogen atom is prepared by reaction of an amine of Formula IIa and an intermediate of Formula IIIa:

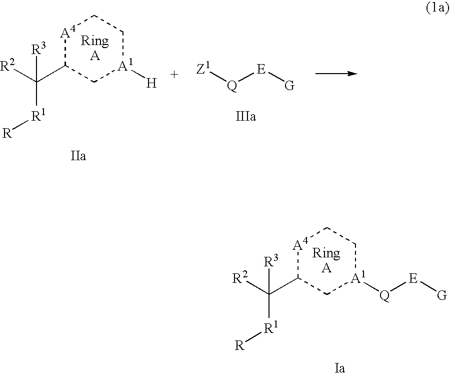

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula IIa in which A¹ is a nitrogen atom are prepared from intermediates of Formula IVa:

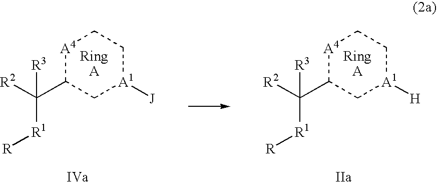

wherein J is an amine protecting group, including carbamate, amide and sulfonamide protecting groups known in the art (T. W. Greene and P. G. M. Wuts “Protective Groups in Organic Synthesis” John Wiley & Sons, Inc., New York 1999).

Intermediates of Formula IVa wherein R³═OH are prepared from ketone intermediates of formula Va by addition of an organometallic reagent of formula VIa, where M is for example Li, MgCl, MgBr, or MgI, to the carbonyl group of Va:

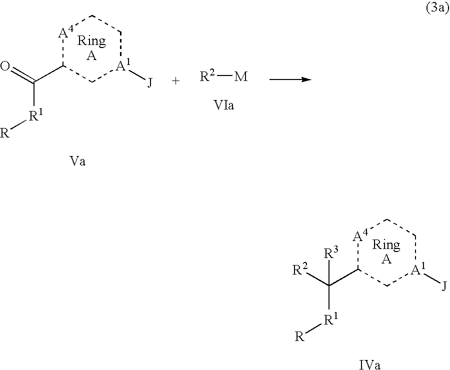

Intermediates of Formula IVa wherein R³═H and R² is a group attached by an ether linkage are prepared from alcohol intermediates of formula VIIa by reaction with an alkylating agent under basic conditions or by reaction with an alcohol under acidic conditions.

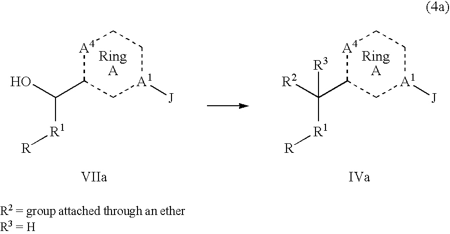

Alcohol intermediates of formula VIIa are prepared by reduction of ketone intermediates of formula Va using reagents known in the art (Handbook of Reagents for Organic Synthesis: Oxidizing and Reducing Reagents Ed. S. D. Burke and R. L. Danheiser, John Wiley & Sons, New York, 1999):

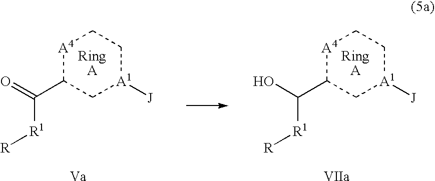

or by addition of an organometallic reagent of formula VIIIa, wherein M is, for example Li, MgCl, MgBr, or MgI, to an aldehyde of Formula IXa:

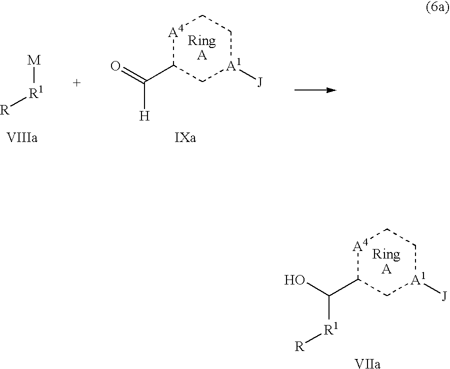

Ketone intermediates of formula Va are prepared by the addition of an organometallic reagent of formula VIIIa, wherein M is Li, MgCl, MgBr, MgI, to a carboxylic acid derivative of formula Xa wherein Z² is an alkoxy, dialkylamino group, or an N-alkoxy-N-alkylamino group:

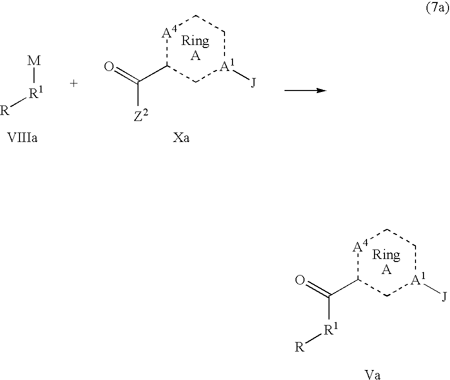

Intermediates of formula Va are also prepared by oxidation of alcohol intermediates of formula VIIa using reagents known in the art (Handbook of Reagents for Organic Synthesis: Oxidizing and Reducing Reagents Ed. S. D. Burke and R. L. Danheiser, John Wiley & Sons, New York, 1999):

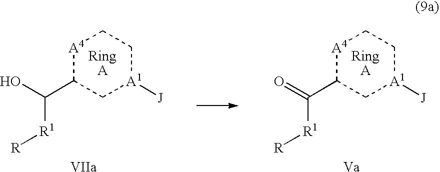

Intermediates of Formula IVa, wherein the R is group attached to R¹ through an ether linkage, are also prepared by alkylation of intermediates of formula XIIIa, in which Z³ is a hydroxyl group with alkylating agents of formula XIVa, wherein X is a halogen, alkanesulfonate, haloalkanesulfonate, or arenesulfonate leaving group:

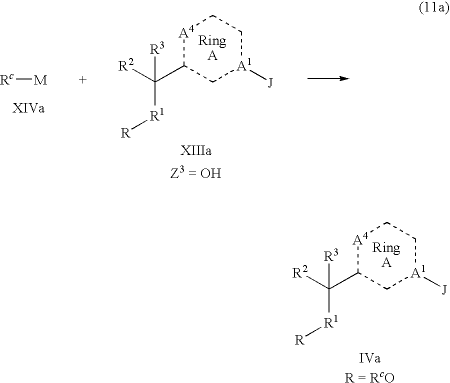

The intermediates of Formula XIIIa used in reaction schemes 10a and 11a are available by processes analogous to those described for IVa (reaction schemes 3a and 4a).

Intermediates of Formula IIIa, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XVa:

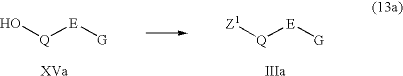

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula IIIa are often prepared and used in situ without isolation.

In the second process of the invention, a compound of Formula Ia, in which a nitrogen atom that is part of E is attached to Q, is prepared by reaction of an intermediate of Formula XVIIIa and an amine of Formula XVIa:

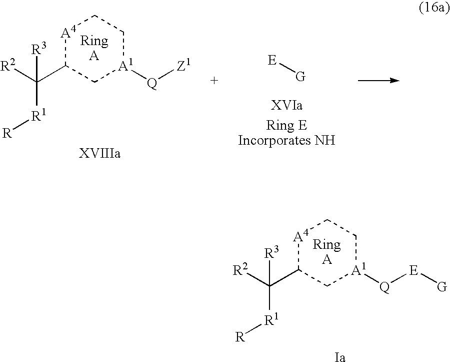

wherein Z¹ is as defined above.

Intermediates of Formula XVIIIa wherein Q is attached to a nitrogen atom of ring A and Q is Q1, Q4, Q5, Q6, Q8, Q9, or Q10 are prepared from amine intermediates of Formula IIa and intermediates of Formula XVIIa wherein Z¹ is halide, alkanesulfonate, haloalkanesulfonate, carboxylate, arylsulfonate, aryloxy, heteroaryloxy, azole, azolium salt, alkoxy, alkylthio, or arylthio:

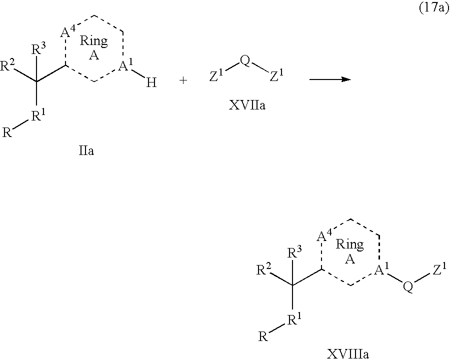

In the fifth process of the invention, a compound of Formula Ia, in which R is an alkoxy, cycloalkoxy, cycloalkylalkoxy or arylalkoxy group, is prepared by reaction of an alkylating agent of Formula XIVa, in which Z³ is chloride, bromide, iodide, methanesulfonate, arenesulfonate or trifluoromethanesulfonate and Rc is an alkyl, cycloalkyl, cycloalkylalkyl or arylalkyl group, with a hydroxy compound of Formula XXIIa:

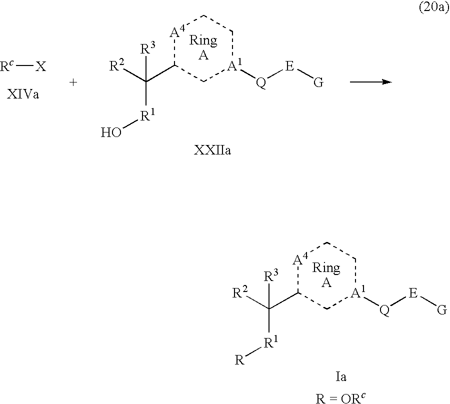

Intermediates of Formula XXIIa are prepared by routes analogous to those shown for compounds of Formula Ia in reaction schemes 1a and 16a.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art. During the course of preparing aryl 3-piperidinyl ketones, as described in the following protocols (e.g. Preparations 3 and 4), racemization of the stereocenter adjacent to the carbonyl group can occur and was specifically observed to occur during the preparation of (R)-tert-butyl 3-(6-chloro-3′-ethylbiphenylcarbonyl)piperidine-1-carboxylate. In this case, the racemic product was detected when the reaction mixture was allowed to stir at room temperature for prolonged times (e.g. overnight) but was not observed when the ketone forming reaction was quenched at −78 ° C. (by addition of aqueous ammonium chloride). When racemization does occur, the resulting stereoisomers may be resolved using conventional methods well known to those skilled in the art. Accordingly, it will be appreciated by those skilled in the art, that in the following Experimental section, any identification of a specific stereoisomer (e.g., assignment of configuration of a chiral center) in a final or intermediate product compound name or structure is to be understood to represent the intended relative or absolute configuration of that chiral center, but not necessarily the only stereoisomer obtained.

The following abbreviations have the indicated meanings

| Abbreviation | Meaning |
|---|---|
| Aq | aqueous |
| Boc | tert-butoxy carbonyl or t-butoxy carbonyl |
| (Boc)₂O | di-tert-butyl dicarbonate |
| brine | saturated aqueous NaCl |
| CH₂Cl₂ | methylene chloride |
| CH₃CN or MeCN | acetonitrile |
| Cpd | compound |
| d | day |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC.HCl | 1-[3-(dimethylamino)propyl]-3-ethylcarbodiimide |
| | hydrochloride |
| equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3- |
| | tetramethyluronium |
| | hexafluorophosphate |
| KHMDS | potassium hexamethyldisilazane |
| LAH or LiAlH₄ | lithium aluminum hydride |
| LC-MS | liquid chromatography-mass spectroscopy |
| LHMDS | lithium hexamethyldisilazane |
| Me | methyl |
| MeCN | acetonitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin- |
| | 2,5-dione |
| TFA | trifluoroacetic acid |
| THF | tetrahydrofuran |
| TMSCl | chlorotrimethylsilane or trimethylsilyl chloride |
| t_{R} | retention time |

### LC-MS Methods

Method 1 [Instrument 1]: Analytical LC-MS was conducted on an Agilent 1100 Series LC/MSD SL or VL using electrospray positive [ES+ve to give MH⁺] equipped with a Sunfire C₁₈ 5.0 μm column (3.050 mm×50 3.0 mm, i.d.), eluting with 0.05% TFA in water (solvent A) and 0.05% TFA in acetonitrile (solvent B), using the following elution gradient 10%-99% (solvent B) over 3.0 min and holding at 99% for 1.0 min at a flow rate of 1.0 ml/min.

Method 2 [Instrument 2]: Analytical LC-MS was conducted on an PE Sciex API 150 single quadrupole mass spectrometer using electrospray positive [ES+ve to give MH+] equipped with a Aquasil C18 5 μm column (1 mm×40 mm), eluting with 0.02% TFA in water (solvent A) and 0.018% TFA in acetonitrile (solvent B), using the following elution gradient 4.5%-90% (solvent B) over 3.2 min and holding at 90% for 0.4 min at a flow rate of 0.3 ml/min.

Method 3 [LC-MS (3 min)]: Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

The following procedures describe preparation of intermediates used in the synthesis of compounds of Formula I

### Preparation 1—Weinreb Amide

### (R)-tert-butyl 3-(N-methoxy-N-methylcarbamoyl)piperidine-1-carboxylate

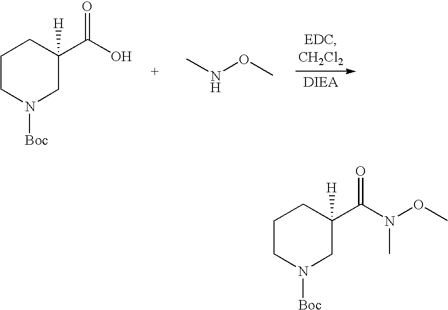

(R)-1-(tert-butoxycarbonyl)piperidine-3-carboxylic acid (25 g, 0.11 mol, 1.0 equiv), N,O-dimethylhydroxylamine hydrochloride, (10.5 g, 0.14 mol, 1.25 equiv), EDC.HCl (26.3 g, 0.14 mol, 1.25 equiv) and DIEA (48 mL, 0.28 mol, 2.5 equiv) were dissolved in CH₂Cl₂ (400 mL) and stirred overnight at rt. The reaction mixture was diluted with EtOAc, washed with 5% aq HCl (2×150mL), satd aq NaHCO₃ (150 mL), brine (100 mL), and dried over Na₂SO₄. Concentration afforded (R)-tert-butyl 3-(N-methoxy-N-methylcarbamoyl)-piperidine-1-carboxylate (24.42 g, 82%) as a clear oil.

### Preparation 2—Biaryl Syntheses

### a) 6-Bromo-2-fluoro-3′-methylbiphenyl

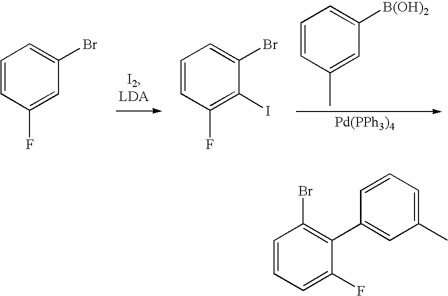

### Step 1. 1-Bromo-3-fluoro-2-iodobenzene

To a solution of diisopropylamine (76 mL, 0.4 mol) in dry THF (664 mL) and n-hexane (220 mL) was added 2.5 M n-BuLi (160 mL. 0.4 mol) dropwise at −78° C. during a period of 1 h. The mixture was stirred for 1 h at −78° C. Then a solution of 1-bromo-3-fluoro-benzene (69 g, 0.4 mol) in dry THF (300 mL) at −78° C. was added to the above mixture dropwise. After stirring for an additional 1 h at −78° C., the mixture was added a solution of iodine (101 g, 0.4 mol) in dry THF (400 mL) dropwise at −78° C. The temperature was raised from −78° C. to rt during 2 h. After stirring for 18 h at rt, the mixture was concentrated in vacuo to give crude product (120 g) which was distilled under reduced pressure to afford 1-bromo-3-fluoro-2-iodobenzene (110 g). ¹H NMR (400 MHz, DMSO): 7.24-7.19 (t, 1H), 7.38-7.32 (m, 1H), 7.55-7.53 (d, 1H).

### Step 2. 6-Bromo-2-fluoro-3′-methylbiphenyl

Pd(Ph₃P)₄ in a 500-mL round-bottom flask under N₂ atmosphere was treated sequentially with a solution of 1-bromo-3-fluoro-2-iodo-benzene (30 g, 0.1 mol) in toluene (250 mL), a solution of 2N aq Na₂CO₃ (200 mL) and 3-methyl phenylboronic acid in ethanol (62 mL). This mixture was heated at reflux under N₂ for 12 h, then cooled to rt. The mixture was partitioned between water and EtOAc. The combined organic layers were washed with brine, dried over MgSO₄, evaporated and purified by column chromatography to give 6-bromo-2-fluoro-3′-methyl-biphenyl (12 g). ¹H NMR (400 MHz, CD₃OD): 7.03 (m, 2H), 7.48-7.04 (m, 4H), 7.50 (d, 1H).

### b) 6-Bromo-2-chloro-3′-methyl-biphenyl

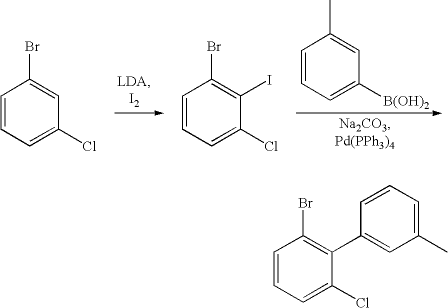

### Step 1. 1-bromo-3-chloro-2-iodobenzene

To a solution of diisopropylamine (76 mL, 0.4 mol) in anhydrous THF (664 mL) and n-hexane (220 mL) was added 2.5 M n-BuLi (160 mL, 0.4 mol) dropwise at −78° C. over 1 h. The mixture was stirred for 1 h at −78° C. and a solution of 1-bromo-3-chlorobenzene (76 g, 0.4 mol) in anhydrous THF (300 mL) was added dropwise at −78° C. After stirring for an additional 1 h at the same temperature, a solution of iodine (101 g, 0.4 mol) in anhydrous THF (400 mL) was added dropwise at −78° C. The temperature was raised from −78° C. to rt during 2 h. After stirring for 18 h at rt, the mixture was concentrated in vacuo to give the crude product (120 g) which was distilled under reduced pressure to give 1-bromo-3-fluoro-2-iodobenzene (115 g, 91%). ¹H NMR (400 MHz, CDCl₃): 7.12-7.18 (t, 1H), 7.35-7.41 (dd, 1H), 7.49-7.54 (dd, 1H); MS (E/Z): 317 (M+H⁺)

### Step 2. 6-bromo-2-chloro-3′-methyl-biphenyl

A 500-mL round-bottom flask under N₂ atmosphere was charged sequentially with Pd(Ph₃P)₄, 1-bromo-3-fluoro-2-iodobenzene (10 g, 0.032 mol) in toluene (80 mL), 2N aqueous sodium carbonate (55 mL) and 3-methylphenylboronic acid (5.16 g, 0.032 mol) dissolved in ethanol (40 mL). This mixture was heated at reflux under N₂ for 12 h and cooled to rt. The mixture was partitioned between water and EtOAc. The combined organic layers were washed with brine, dried over MgSO₄, and concentrated. The residue was purified by column chromatography to give 6-bromo-2-chloro-3′-methyl-biphenyl (6 g, 67%). ¹H NMR (400 MHz, CD₃OD): 6.90-7.00 (t, 2H), 7.14-7.24 (m, 2H), 7.26-7.33 (t, 1H), 7.44-7.50 (d, 1H), 7.58-7.62 (d, 1H); MS (E/Z): 281 (M+H⁺)

The following biaryls were prepared from aryl halides and the boronic acids indicated using the procedures described in Preparations 14a Step 2 and 14b Step 2:

| Biaryl | Aryl halide | Boronic acid |
|---|---|---|
| 2-bromo-6-chloro-3′- | 1-bromo-3-chloro- | 3-ethylphenylboronic acid |
| ethylbiphenyl | 2-iodobenzene | |
| 2-bromo-3′-ethyl-6- | 1-bromo-3-fluoro- | 3-ethylphenylboronic acid |
| fluorobiphenyl | 2-iodobenzene | |

### Preparation 3

### Methyl {4-(6-chloro-3′-methyl-2-biphenylyl)-4-hydroxy-4-[(3R)-3-piperidinyl]butyl}carbamate

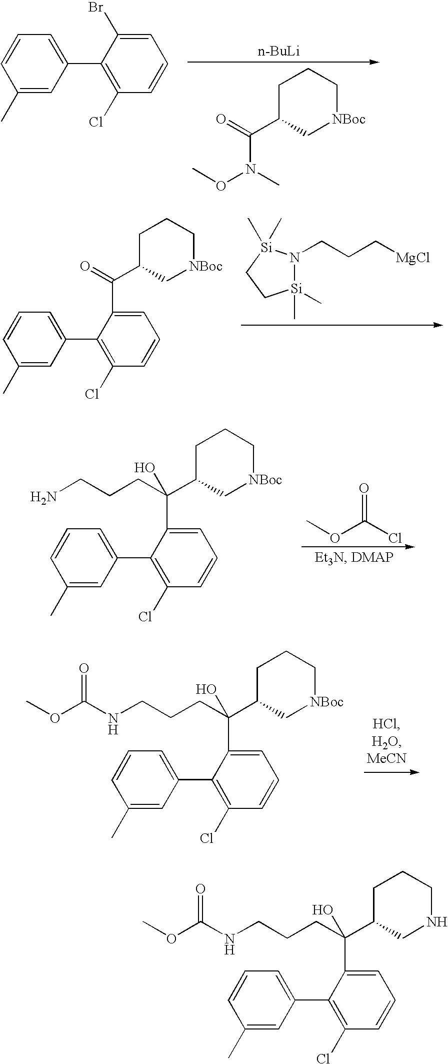

Step 1. (R)-tert-butyl 3-(6-chloro-3′-methylbiphenylcarbonyl)piperidine-1-carboxylate: To a solution of 6-bromo-2-fluoro-3′-methylbiphenyl (2 g, 7.14 mmol) in anhydrous THF (30 mL) cooled to −78° C. was added dropwise a solution of 1.6 M of n-BuLi in hexane (4.46 mL). The reaction mixture was stirred at −78° C. for 1 h and a solution of (R)-tert-butyl 3-(methoxy(methyl)carbamoyl)piperidine-1-carboxylate (1.94 g, 7.14 mmol) in anhydrous THF (20 mL) was added. The mixture was allowed to warm to rt and stirred overnight. The mixture was quenched with satd aq NH₄Cl (40 mL) and extracted with EtOAc (40 mL). The combined organic layers were dried over Na₂SO₄ and concentrated to give crude product, which was purified by flash column chromatography to afford (R)-tert-butyl 3-(6-chloro-3′-methylbiphenylcarbonyl)piperidine-1-carboxylate (1 g, 34%). ¹H NMR (400 MHz, CD₃OD): 0.80-1.20 (m, 8H), 1.30 (s, 1H), 1.40 (s, 1H), 1.40-1.60 (m, 2H), 2.00-2.18 (s, 1H), 2.30-2.40 (s, 3H), 2.60-2.80 (m, 2H), 3.50-3.80 (m, 2H), 7.00-7.15 (s, 2H), 7.20-7.30 (d, 1H), 7.30-7.40 (t, 2H), 7.39-7.48 (t, 1H), 7.60-7.70 (d, 1H); MS (E/Z): 414 (M+H⁺)

Step 2. 1,1-dimethylethyl (3R)-3-[4-amino-1-(6-chloro-3′-methyl-2-biphenylyl)-1-hydroxybutyl]-1-piperidinecarboxylate: To a solution of (R)-tert-butyl 3-(6-chloro-3′-methylbiphenylcarbonyl)piperidine-1-carboxylate (800 mg, 1.94 mmol) in anhydrous THF (15 mL) cooled to −78° C. was added dropwise a solution of 2 M (3-(2,2,5,5-tetramethyl-1,2,5-azadisilolidin-1-yl)propyl)magnesium chloride in THF (0.968 mL, 1.94 mmol). After addition, the reaction mixture was allowed to warm slowly to rt while stirring overnight. The mixture was quenched with satd aq NH₄Cl (15 mL) and extracted with CH₂Cl₂ (3×). The combined organic layers were dried over Na₂SO₄ and concentrated to give crude 1,1-dimethylethyl (3R)-3-[4-amino-1-(6-chloro-3′-methyl-2-biphenylyl)-1-hydroxybutyl]-1-piperidinecarboxylate (900 mg), which was used in the next step without further purification.

Step 3. 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate: To a solution of 1,1-dimethylethyl (3R)-3-[4-amino-1-(6-chloro-3′-methyl-2-biphenylyl)-1-hydroxybutyl]-1-piperidinecarboxylate (800 mg, 1.69 mmol) in anhydrous CH₂Cl₂ (15 mL) were added 4-dimethyaminopyridine (1.24 g, 10.17 mmol) and Et₃N (2.35 mL, 16.95 mmol). The mixture was cooled with an ice bath and methyl chloroformate (0.65 mL, 8.47 mmol) in CH₂Cl₂ (5 mL) was added. The reaction mixture was allowed to warm slowly to rt while stirring overnight. The solvent was removed in vacuo and the residue was purified by column chromatography to afford 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (700 mg, 78%). ¹H NMR (400 MHz, CD₃OD): 1.00-1.70 (m, 17H), 2.30-2.50 (d, 3H), 2.50-2.70 (s, 1H), 2.90-2.31 (m, 2H), 3.50-3.52 (m, 3H), 3.80-4.20 (m, 2H), 6.0-7.15 (m, 3H), 7.15-7.40 (m, 3H), 7.50-7.70 (m, 1H); MS (E/Z): 531 (M+H⁺)

Step 4. Methyl {4-(6-chloro-3′-methyl-2-biphenylyl)-4-hydroxy-4-[(3R)-3-piperidinyl]butyl}carbamate: To a solution of 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (600 mg, 1.13 mg) in CH₃CN (18 mL) was added 2N aq HCl (15 mL) and the reaction mixture was vigorously stirred overnight at rt. The solvents were removed in vacuo to give methyl {4-(6-chloro-3′-methyl-2-biphenylyl)-4-hydroxy-4-[(3R)-3-piperidinyl]butyl}carbamate as its hydrochloride salt (500 mg, 95.8%). ¹H NMR (400 MHz, CD₃OD): 1.00-1.20 (m, 1H), 1.30-1.80 (m, 8H), 1.80-2.00 (m, 2H), 2.40-2.50 (d, 3H), 2.75-2.90 (t, 1H), 2.90-3.05 (m, 3H), 3.05-3.12 (t, 1H), 3.20-3.30 (m, 1H), 3.30-3.40 (m, 1H), 3.60-3.70 (d, 4H), 6.90-6.98 (d, 1H), 7.00-7.12 (m, 1H), 7.25-7.50 (m, 4H), 7.75-7.85 (d, 1H); MS (E/Z): 431 (M+H⁺)

The following piperidines were prepared using procedures analogous to those described above:

Methyl 4-(6-chloro-3′-ethylbiphenyl-2-yl)-4-hydroxy-4-((R)-piperidin-3-yl)butylcarbamate using 2-bromo-6-chloro-3′-ethylbiphenyl in Step 1.

Methyl 4-(3′-ethyl-6-fluorobiphenyl-2-yl)-4-hydroxy-4-((R)-piperidin-3-yl)butylcarbamate using 2-bromo-6-fluoro-3′-ethylbiphenyl in Step 1.

N-{4-(6-chloro-3′-methyl-2-biphenylyl)-4-hydroxy-4-[(3R)-3-piperidinyl]butyl}acetamide using 2-bromo-6-chloro-3′methylbiphenyl in Step 1 and acetyl chloride instead of methyl chloroformate in Step 3.

### Preparation 4

### (5-methoxy-1-(2-phenoxyphenyl)-1-((R)-piperidin-3-yl)pentan-1-ol

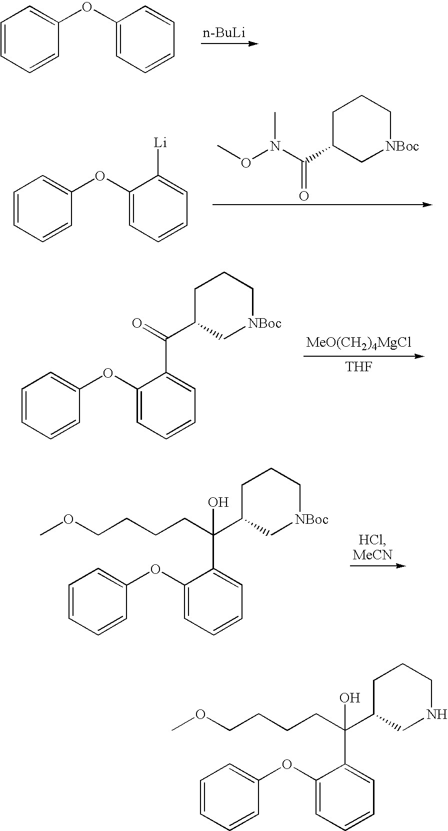

### Step 1. 2-(Phenoxy)phenyllithium

To a solution of diphenyl ether (8.60 g, 50.0 mmol) in Et₂O (75 mL) was added n-BuLi (1.6 M in hexane, 32.8 mL, 52.5 mmol). The mixture was refluxed for 48 h, and the resulting solution of 2-(phenoxy)phenyllithium was used in the next step without any further analysis.

### Step 2. (3R)-1-(tert-butoxycarbonyl)-3-(2-phenoxybenzoyl)piperidine

To a solution of (R)-tert-butyl 3-(N-methoxy-N-methylcarbamoyl)piperidine-1-carboxylate (4.40 g, 16.2 mmol) in anhydrous THF (18 mL) at −10° C., was added dropwise the solution of 2-phenoxyphenyllithium prepared in Step 1 (80 mL, 32 mmol). The mixture was then warmed to rt, and stirred until no starting material remained (˜30 min). The reaction was quenched with 1 N HCl (˜30 mL) and extracted with Et₂O (4×10 mL). The combined organic layers were washed with satd aq NaHCO₃ and brine, and dried over Na₂SO₄. The solvent was removed to give (3R)-1-(tert-butoxycarbonyl)-3-(2-phenoxybenzoyl)piperidine (7.44 g, quantitative).

### Step 3. (3R)-tert-Butyl 3-(1-hydroxy-5-methoxy-1-(2-phenoxyphenyl)pentyl)piperidine-1-carboxylate

To a solution of (3R)-1-(tert-butoxycarbonyl)-3-(2-phenoxybenzoyl)piperidine (6.17 g, 16.2 mmol) in THF (30 mL) at −10° C. was added dropwise 2.54 M 4-methoxybutylmagnesium chloride in THF (15 mL, 38 mmol). The resulting solution was warmed to rt slowly, and stirred over night. The reaction was quenched with satd NH₄Cl (10 mL) and extracted with Et₂O (4×10 mL). The combined organic layers were washed with water and brine. The solvent was removed and the residue was purified by flash chromatography to give (3R)-tert-Butyl 3-(1-hydroxy-5-methoxy-1-(2-phenoxyphenyl)pentyl)piperidine-1-carboxylate (1.97 g, 26% from (R)-tert-butyl 3-(N-methoxy-N-methylcarbamoyl)piperidine-1-carboxylate).

### Step 4. (5-methoxy-1-(2-phenoxyphenyl)-1-((R)-piperidin-3-yl)pentan-1-ol

To a solution of (R)-tert-butyl 3-((S)-1-hydroxy-5-methoxy-1-(2-phenoxyphenyl)pentyl)piperidine-1-carboxylate (1.97 g, 4.19 mmol) in MeCN (100 mL) was added 2 N aq HCl (100 mL) slowly at rt. The resulting solution was stirred at rt until no starting material remained (˜16 h), basified to pH=10 with 10 N aq NaOH, and evaporated under reduced pressure to remove MeCN. The aq layer was extracted with CH₂Cl₂ (4×10 mL). The combined organic layers were washed with brine and dried over Na₂SO₄. The solvent was removed in vacuo to afford (5-methoxy-1-(2-phenoxyphenyl)-1-((R)-piperidin-3-yl)pentan-1-ol (1.56 g, quantitative) as a free amine.

### Preparation 5 Piperidines from Weinreb Amides and Bromobiaryls

### 1-(2′-chloro-2-biphenylyl)-5-(methyloxy)-1-[(3R)-3-piperidinyl]-1-pentanol

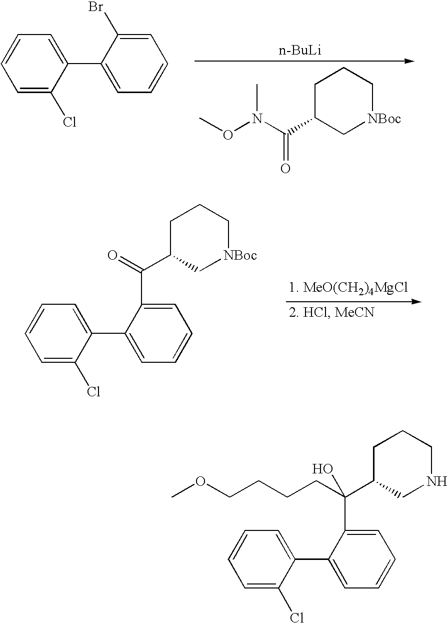

Step 1. (3R)-1-(tert-butoxycarbonyl)-3-((2-(2-chlorophenyl))benzoyl)piperidine: To a solution of 2′-bromo-2-chloro-biphenyl (5.34 g, 20 mmol) in anhydrous THF (50 mL) cooled to −78° C. was added dropwise a solution of 1.6 M n-BuLi in hexane (12.5 mL, 20 mmol). The reaction mixture was stirred at −78° C. for 1 h and a solution of (R)-tert-butyl 3-(N-methoxy-N-methylcarbamoyl)-piperidine-1-carboxylate (5.44 g, 20 mmol) in anhydrous THF (50 mL) was added. The mixture was allowed to warm to rt and stirred overnight. The mixture was quenched with satd aq NH₄Cl (100 mL) and extracted with EtOAc (3×75 mL). The combined organic layers were dried over Na₂SO₄ and concentrated to give the crude product, which was purified by flash column chromatography to afford (3R)-1-(tert-butoxycarbonyl)-3-((2-(2-chlorophenyl))benzoyl)piperidine (4.43 g, 55%).

Step 2. 1,1-dimethylethyl (3R)-3-[1-(2′-chloro-2-biphenylyl)-1-hydroxy-5-(methyloxy)pentyl]-1-piperidinecarboxylate: A 250 mL three-necked flask was charged with magnesium turning (2.88 g, 0.12 mol) and a small crystal of iodine. The flask was evacuated and refilled with N₂. A solution of 1-chloro-4-methoxybutane (15 g, 0.12 mol) in THF (60 ml) was added dropwise to the above mixture. After heating under reflux for 2 h most of magnesium had been consumed and the Grignard solution was cooled to rt. A 250 mL three-necked flask was charged with (3R)-1-(tert-butoxycarbonyl)-3-((2-(2-chlorophenyl))benzoyl)piperidine (4.43 g, 11 mmol) and THF (50 mL), evacuated and refilled with N₂. The mixture was cooled in a dry ice-acetone bath and the Grignard reagent was added dropwise. The mixture was allowed to warm slowly to rt and stirred overnight. The mixture was quenched with satd aq NH₄Cl (100 mL) and extracted with EtOAc. The combined organic layers were dried over Na₂SO₄ and concentrated to give the crude product which was purified by flash column chromatography to afford pure 1,1-dimethylethyl (3R)-3-[1-(2′-chloro-2-biphenylyl)-1-hydroxy-5-(methyloxy)pentyl]-1-piperidinecarboxylate (2.5 g, 47%).

Step 3. 1-(2′-chloro-2-biphenylyl)-5-(methyloxy)-1-[(3R)-3-piperidinyl]-1-pentanol: The Boc protecting group was removed using the protocol described in Preparation 4 Step 4.

The following piperidines were prepared using procedures analogous to those described above:

1-(6-chloro-3′-ethyl-2-biphenylyl)-5-(methyloxy)-1-[(3R)-3-piperidinyl]-1-pentanol using 2-bromo-6-chloro-3′-ethylbiphenyl in Step 1.

### Preparation 6

### lithium {4-[(2-nitrophenyl)sulfonyl]-2-morpholinyl}acetate

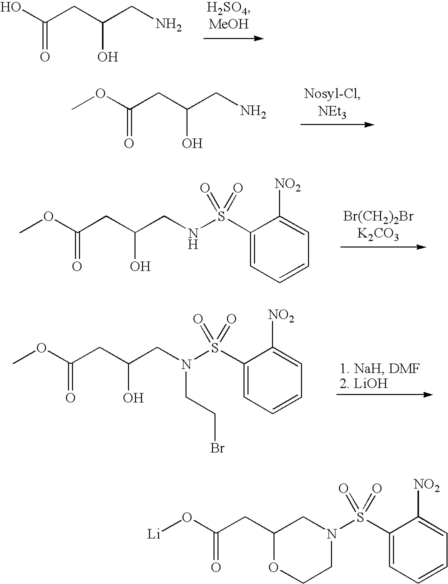

### Step 1. Methyl 4-amino-3-hydroxybutanoate

A solution of 4-amino-3-hydroxybutanoic acid (10.0 g, 83.94 mmol) in 40 mL of MeOH at 25° C. was treated with concentrated H₂SO₄ (3 mL) and the mixture was stirred and heated at 65° C. overnight before being cooled to 0° C. and basified by the addition of solid KHCO₃. The suspension was filtered thru celite and concentrated to give a gum, which was dissolved in 80 mL of acetonitrile and slowly treated with 21 mL of 4N HCl in dioxane solution. The resulting solution was concentrated under reduced pressure to give methyl 4-amino-3-hydroxybutanoate as an oil.

### Step 2. Methyl 3-hydroxy-4-{[(2-nitrophenyl)sulfonyl]amino}butanoate

A solution of methyl 4-amino-3-hydroxybutanoate (4.0 g, 23.65 mmol) in 35 mL of CH₂Cl₂ at 0° C. was treated with Et₃N (9.9 mL, 70.95 mmol) and a solution of 2-nitrosulfonyl chloride in 10 mL of CH₂Cl₂, and the mixture was stirred at 0° C. for 30 min before being quenched with the addition of saturated aqueous KHCO₃ (25 mL). The organic layer was separated, dried (MgSO₄), concentrated under reduced pressure, and subjected to flash chromatography to give methyl 3-hydroxy-4-{[(2-nitrophenyl)sulfonyl]amino}butanoate as a light brown oil (3.07 g, 41%). ESI-MS (m/z): 341.0 (M+Na⁺).

### Step 3. Methyl 4-{(2-bromoethyl)[(2-nitrophenyl)sulfonyl]amino}-3-hydroxybutanoate

A solution of methyl 3-hydroxy-4-{[(2-nitrophenyl)sulfonyl]amino}butanoate (0.5 g, 1.57 mmol) in 8 mL of DMF at 25° C. was treated with 1,2-dibromoethane (1.35 mL, 15.7 mmol) and K₂CO₃ (0.43 g, 3.14 mmol) and the mixture was stirred overnight before being quenched with the addition of water and extracted with EtOAc. The organic extract was dried (MgSO₄) and concentrated under reduced pressure to give methyl 4-{(2-bromoethyl)[(2-nitrophenyl)sulfonyl]amino}-3-hydroxybutanoate as a solid. ESI-MS (m/z): 425.0 (M+H⁺).

### Step 4. Methyl {4-[(2-nitrophenyl)sulfonyl]-2-morpholinyl}acetate

A solution of methyl 4-{(2-bromoethyl)[(2-nitrophenyl)sulfonyl]amino}-3-hydroxybutanoate (0.45 g, 1.0 mmol) in 1 mL of DMF at 0° C. was treated with NaH (0.030 g, 1.2 mmol) and the mixture was stirred for 10 min before being quenched with the addition of water (5 mL) and extracted with EtOAc (3×4 mL). The combined organic extracts were concentrated under reduced pressure and subjected to reverse phase HPLC to give methyl {4-[(2-nitrophenyl)sulfonyl]-2-morpholinyl}acetate as a thick oil (0.015 g, 63%). ESI-MS (m/z): 345.1 (M+H⁺).

### Step 5. Lithium {4-[(2-nitrophenyl)sulfonyl]-2-morpholinyl}acetate

A solution of methyl {4-[(2-nitrophenyl)sulfonyl]-2-morpholinyl}acetate (0.36 g, 1.0 mmol) in 4 mL of MeOH and 1 mL of H₂O at 25° C. was treated with LiOH (0.30 g, 12.6 mmol) and the mixture was stirred for 1 h before being concentrated under reduced pressure to give lithium {4-[(2-nitrophenyl)sulfonyl]-2-morpholinyl}acetate as a brown solid that was used without purification.

The following procedures describe preparation of compounds of Formula I.

### Example 1

### 1-((3R)-1-{[(1R,3S)-3-aminocyclopentyl]carbonyl}-3-piperidinyl)-5-(methyloxy)-1-[2-(phenyloxy)phenyl]-1-pentanol

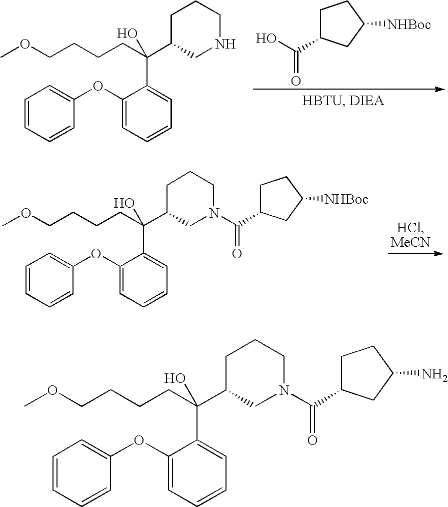

Step 1. ((1R,3S)-3-(tert-butoxycarbonylamino)cyclopentyl)((3R)-3-(1-hydroxy-5-methoxy-1-(2-phenoxyphenyl)pentyl)piperidin-1-yl)methanone: To a solution of 5-methoxy-1-(2-phenoxyphenyl)-1-((R)-piperidin-3-yl)pentan-1-ol (18.5 mg, 0.05 mmol) and (1R,3S)-3-(t-butoxycarbonylamino)cyclopentanecarboxylic acid (12.1 mg, 0.05 mmol) in DMF (0.5 mL) were added DIEA (26 μL. 0.15 mmol), HBTU (19.0 mg, 0.05 mmol), and HOBt (6.8 mg, 0.05 mmol). The resulting solution was stirred at rt for 20 min. Preparative HPLC gave ((1R,3S)-3-(tert-butoxycarbonylamino)cyclopentyl)((3R)-3-(1-hydroxy-5-methoxy-1-(2-phenoxyphenyl)pentyl)piperidin-1-yl)methanone (19.5 mg, 67%) as a oil. LC-MS (3 min) m/z 581 (M+H⁺).

Step 2. ((1R,3S)-3-Aminocyclopentyl)((3R)-3-(1-hydroxy-5-methoxy-1-(2-phenoxyphenyl)pentyl)piperidin-1-yl)methanone: To a stirred solution of ((1R,3S)-3-(tert-butoxycarbonylamino)cyclopentyl)((3R)-3-(1-hydroxy-5-methoxy-1-(2-phenoxyphenyl)pentyl)piperidin-1-yl)methanone (19.5 mg) in MeCN (2 mL) was added 5% aq HCl (2 mL). The resulting solution was stirred at rt until no starting material remained (˜16 h), basified to pH=10 with 10 N aq NaOH, and evaporated under reduced pressure to remove MeCN. The aq layer was extracted with CH₂Cl₂ (4×10 mL). The combined organic layers were washed with brine and dried over Na₂SO₄. The crude product was purified by preparative HPLC to give ((1R,3S)-3-Aminocyclopentyl)((3R)-3-(1-hydroxy-5-methoxy-1-(2-phenoxyphenyl)pentyl)piperidin-1-yl)methanone (I-4A, 17.4 mg) as its TFA salt. ¹H NMR (400 MHz, CD₃OD): 7.64 (m, 1H), 7.38 (m, 2H), 7.08-7.24 (m, 3H), 6.92 (m, 2H), 6.80 (two d, 1H), 4.44, 4.86 (m, 1H), 3.96, 4.26 (m, 1H), 3.68 (m,1H), 3.36, 3.44 (m, 1H), 3.28 (t, 2H), 3.24 (s, 3H), 2.94, 3.14 (m, 1H), 2.63 (m, 1H), 2.40 (m, 1H), 1.8-2.2 (m, 6H), 1.0-1.8 (m, 8H), 0.92 (m, 1H); LC-MS (3 min) m/z 481 (M+H⁺).

((3R)-3-(1-hydroxy-5-methoxy-1-(2-phenoxyphenyl)pentyl)piperidin-1-yl)(piperidin-4-yl)methanone (#3) was prepared following the procedure described above using 1-(tert-butoxycarbonyl)piperidine-4-carboxylic acid in Step 1.

### Example 2

### Methyl (S)-4-(6-chloro-3′-ethylbiphenyl-2-yl)-4-hydroxy-4-((R)-1-(4-((methylamino)methyl)benzoyl)piperidin-3-yl)butylcarbamate

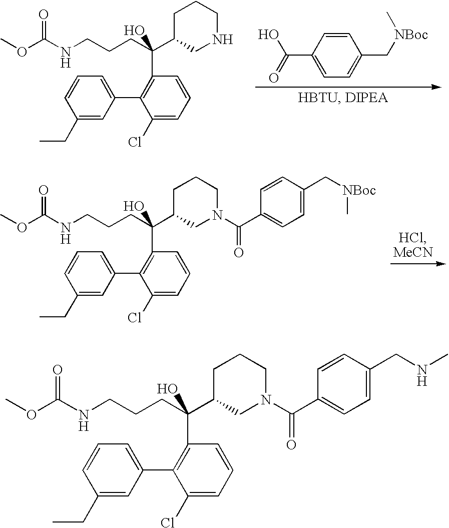

### Step 1. methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate

A solution of methyl (S)-4-(6-chloro-3′-ethylbiphenyl-2-yl)-4-hydroxy-4-((R)-piperidin-3-yl)butylcarbamate (30 mg, 0.07 mmol) in 1 mL of DMF at 25° C. was treated with 4-((tert-butoxycarbonyl(methyl)amino)methyl)benzoic acid (21 mg, 0.08 mmol), i-Pr₂NEt (0.063 mL, 0.37 mmol), and HBTU (30 mg, 0.08 mmol). After 1 h, H₂O was added and the mixture was extracted with EtOAc. The organic extracts were washed (1N HCl, 1N NaOH, H₂O, brine), dried (Na₂SO₄), concentrated under reduced pressure, and subjected to flash chromatography to provide methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate as a colorless oil (24 mg, 51%). MS (m/z) 692.3 (M+H⁺).

### Step 2. methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate

A solution of methyl (S)-4-(6-chloro-3′-ethylbiphenyl-2-yl)-4-hydroxy-4-((R)-1-(4-((N-t-butoxycarbonyl-N-methyl)amino)methyl)benzoyl)piperidin-3-yl)butylcarbamate (24 mg, 0.034 mmol) in 3 mL of CH₃CN at 25° C. was treated with 3 mL of aqueous 2N HCl. After 24 h, the mixture was concentrated under reduced pressure to provide methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate as a white solid (17 mg, 81%). MS (m/z) 592.2 (M+H⁺).

### Example 3

The following piperidines were prepared following procedures analogous to those described in Example 2 using the appropriate amine intermediate and the indicated acid in place of 4-((tert-butoxycarbonyl(methyl)amino)methyl)benzoic acid in Step 1.

| | Product | Acid used in Step 1 |
|---|---|---|
| #1 | methyl 4-(3′-ethyl-6-fluorobiphenyl-2-yl)- | (4R)-1-{[(1,1- |
| | 4-hydroxy-4-((R)-1-((2S,4R)-4- | dimethylethyl)oxy]carbonyl}-4-hydroxy- |
| | hydroxypyrrolidine-2-carbonyl)piperidin- | L-proline |
| | 3-yl)butylcarbamate | |
| #2 | methyl (4-(3′-ethyl-6-fluoro-2- | (3R)-1-{[(1,1- |
| | biphenylyl)-4-hydroxy-4-{(3R)-1-[(3R)-3- | dimethylethyl)oxy]carbonyl}-3- |
| | pyrrolidinylcarbonyl]-3- | pyrrolidinecarboxylic acid |
| | piperidinyl}butyl)carbamate | |
| #4 | methyl (4-(3′-ethyl-6-fluoro-2- | ((2S)-1-{[(1,1- |
| | biphenylyl)-4-hydroxy-4-{(3R)-1-[(2S)-2- | dimethylethyl)oxy]carbonyl}-2- |
| | pyrrolidinylacetyl]-3- | pyrrolidinyl)acetic acid |
| | piperidinyl}butyl)carbamate | |
| #5 | methyl {4-(3′-ethyl-6-fluoro-2- | 1-{[(1,1-dimethylethyl)oxy]carbonyl}-2- |
| | biphenylyl)-4-hydroxy-4-[(3R)-1-(2- | piperidinecarboxylic acid |
| | piperidinylcarbonyl)-3- | |
| | piperidinyl]butyl}carbamate | |
| #6 | methyl (4-(3′-ethyl-6-fluoro-2- | (3R)-1-{[(1,1- |
| | biphenylyl)-4-hydroxy-4-{(3R)-1-[(3R)-3- | dimethylethyl)oxy]carbonyl}-3- |
| | piperidinylcarbonyl]-3- | piperidinecarboxylic acid |
| | piperidinyl}butyl)carbamate | |
| #7 | methyl {4-(3′-ethyl-6-fluoro-2- | 1-{[(1,1-dimethylethyl)oxy]carbonyl}-4- |
| | biphenylyl)-4-hydroxy-4-[(3R)-1-(4- | piperidinecarboxylic acid |
| | piperidinylcarbonyl)-3- | |
| | piperidinyl]butyl}carbamate | |
| #8 | methyl {4-(3′-ethyl-6-fluoro-2- | (1-{[(1,1-dimethylethyl)oxy]carbonyl}-4- |
| | biphenylyl)-4-hydroxy-4-[(3R)-1-(4- | piperidinyl)acetic acid |
| | piperidinylacetyl)-3- | |
| | piperidinyl]butyl}carbamate | |
| #9 | methyl {4-(3′-ethyl-6-fluoro-2- | (1-{[(1,1-dimethylethyl)oxy]carbonyl}-3- |
| | biphenylyl)-4-hydroxy-4-[(3R)-1-(3- | piperidinyl)acetic acid |
| | piperidinylacetyl)-3- | |
| | piperidinyl]butyl}carbamate | |
| #10 | methyl (4-(3′-ethyl-6-fluoro-2- | 3-(1-{[(1,1-dimethylethyl)oxy]carbonyl}- |
| | biphenylyl)-4-hydroxy-4-{(3R)-1-[3-(4- | 4-piperidinyl)propanoic acid |
| | piperidinyl)propanoyl]-3- | |
| | piperidinyl}butyl)carbamate | |
| #11 | methyl (4-(3′-ethyl-6-fluoro-2- | 3-(1-{[(1,1-dimethylethyl)oxy]carbonyl}- |
| | biphenylyl)-4-hydroxy-4-{(3R)-1-[3-(2- | 2-piperidinyl)propanoic acid |
| | piperidinyl)propanoyl]-3- | |
| | piperidinyl}butyl)carbamate | |
| #12 | methyl (4-(3′-ethyl-6-fluoro-2- | 1-(N-{[(1,1- |
| | biphenylyl)-4-{(3R)-1-[(1-glycyl-4- | dimethylethyl)oxy]carbonyl}glycyl)-4- |
| | piperidinyl)carbonyl]-3-piperidinyl}-4- | piperidinecarboxylic acid |
| | hydroxybutyl)carbamate | |
| #13 | methyl (4-(3′-ethyl-6-fluoro-2- | 1-(1-{[(1,1-dimethylethyl)oxy]carbonyl}- |
| | biphenylyl)-4-hydroxy-4-{(3R)-1-[1-(4- | 4-piperidinyl)-L-proline |
| | piperidinyl)-L-prolyl]-3- | |
| | piperidinyl}butyl)carbamate | |
| #14 | methyl {4-(3′-ethyl-6-fluoro-2- | N-{[(1,1-dimethylethyl)oxy]carbonyl}-L- |
| | biphenylyl)-4-hydroxy-4-[(3R)-1-(L- | phenylalanyl-L-proline |
| | phenylalanyl-L-prolyl)-3- | |
| | piperidinyl]butyl}carbamate | |
| #15 | methyl [4-(3′-ethyl-6-fluoro-2- | [4-(2-hydroxyethyl)-1-piperazinyl]acetic |
| | biphenylyl)-4-hydroxy-4-((3R)-1-{[4-(2- | acid |
| | hydroxyethyl)-1-piperazinyl]acetyl}-3- | |
| | piperidinyl)butyl]carbamate | |
| #17 | methyl [4-(6-chloro-3′-methyl-2- | L-dihydroorotic acid |
| | biphenylyl)-4-((3R)-1-{[(4S)-2,6- | |
| | dioxohexahydro-4-pyrimidinyl]carbonyl}- | |
| | 3-piperidinyl)-4-hydroxybutyl]carbamate | |
| #18 | methyl [4-(6-chloro-3′-methyl-2- | D-dihydroorotic acid |
| | biphenylyl)-4-((3R)-1-{[(4R)-2,6- | |
| | dioxohexahydro-4-pyrimidinyl]carbonyl}- | |
| | 3-piperidinyl)-4-hydroxybutyl]carbamate | |
| #19 | methyl (4-(3′-ethyl-6-fluoro-2- | 1-(tert-butoxycarbonyl)-4- |
| | biphenylyl)-4-hydroxy-4-{(3R)-1-[(4- | phenylpiperidine-4-carboxylic acid |
| | phenyl-4-piperidinyl)carbonyl]-3- | |
| | piperidinyl}butyl)carbamate | |
| #20 | methyl {4-(3′-ethyl-6-fluoro-2- | 4-(tert-butoxycarbonyl)morpholine-2- |
| | biphenylyl)-4-hydroxy-4-[(3R)-1-(2- | carboxylic acid |
| | morpholinylcarbonyl)-3- | |
| | piperidinyl]butyl}carbamate | |
| #22 | 1-(6-chloro-3′-ethyl-2-biphenylyl)-5- | 2-(1H-tetrazol-5-yl)acetic acid |
| | (methyloxy)-1-[(3R)-1-(1H-tetrazol-5- | |
| | ylacetyl)-3-piperidinyl]-1-pentanol | |
| #23 | methyl {4-(6-chloro-3′-ethyl-2- | 2-(1H-tetrazol-5-yl)acetic acid |
| | biphenylyl)-4-hydroxy-4-[(3R)-1-(1H- | |
| | tetrazol-5-ylacetyl)-3- | |
| | piperidinyl]butyl}carbamate | |
| #24 | N-{4-(6-chloro-3′-methyl-2-biphenylyl)-4- | 2-(1H-tetrazol-5-yl)acetic acid |
| | hydroxy-4-[(3R)-1-(1H-tetrazol-5- | |
| | ylacetyl)-3-piperidinyl]butyl}acetamide | |
| #25 | N-{4-(6-chloro-3′-methyl-2-biphenylyl)-4- | 2-(1H-imidazol-4-yl)acetic acid |
| | hydroxy-4-[(3R)-1-(1H-imidazol-4- | |
| | ylacetyl)-3-piperidinyl]butyl}acetamide | |
| #26 | methyl {4-(3′-ethyl-6-fluoro-2- | isonicotinic acid |
| | biphenylyl)-4-hydroxy-4-[(3R)-1-(4- | |
| | pyridinylcarbonyl)-3- | |
| | piperidinyl]butyl}carbamate | |
| #27 | methyl {4-(3′-ethyl-6-fluoro-2- | 2-(pyridin-4-yl)acetic acid |
| | biphenylyl)-4-hydroxy-4-[(3R)-1-(4- | |
| | pyridinylacetyl)-3- | |
| | piperidinyl]butyl}carbamate | |
| #30 | methyl {4-(6-chloro-3′-ethyl-2- | 2-(tert-butoxycarbonyl)-1,2,3,4- |
| | biphenylyl)-4-hydroxy-4-[(3R)-1-(1,2,3,4- | tetrahydroisoquinoline-6-carboxylic acid |
| | tetrahydro-6-isoquinolinylcarbonyl)-3- | |
| | piperidinyl]butyl}carbamate | |
| #31 | methyl {4-(6-chloro-3′-ethyl-2- | 2-(tert-butoxycarbonyl)-1,2,3,4- |
| | biphenylyl)-4-hydroxy-4-[(3R)-1-(1,2,3,4- | tetrahydroisoquinoline-7-carboxylic acid |
| | tetrahydro-7-isoquinolinylcarbonyl)-3- | |
| | piperidinyl]butyl}carbamate | |

### Example 4

### methyl {4-(6-chloro-3′-ethyl-2-biphenylyl)-4-hydroxy-4-[(3R)-1-(1-piperazinylcarbonyl)-3-piperidinyl]butyl}carbamate (#16)

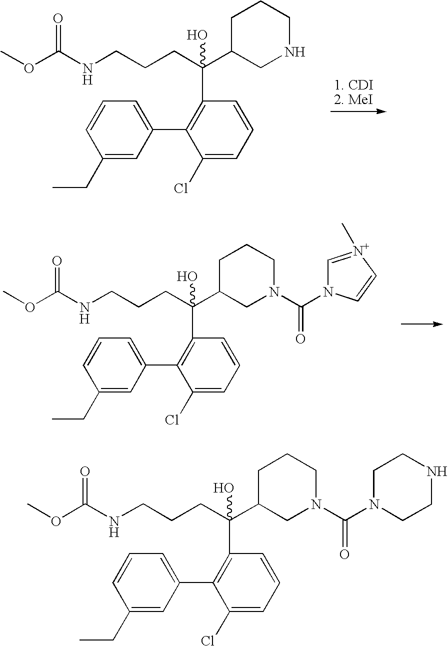

Step 1. 1-{[3-(1-(6-chloro-3′-ethyl-2-biphenylyl)-1-hydroxy-4-{[(methyloxy)carbonyl]amino}butyl)-1-piperidinyl]carbonyl}-3-methyl-1H-imidazol-3-ium: A solution of methyl [4-(6-chloro-3′-ethyl-2-biphenylyl)-4-hydroxy-4-(3-piperidinyl)butyl]carbamate (0.6 g, 1.37 mmol) in 25 mL of CH₂Cl₂ at 25° C. was treated with carbonyl diimidazole (0.22 g, 1.37 mmol) and Et₃N (0.35 mL, 2.5 mmol), and the mixture was stirred overnight before being concentrated under reduced pressure. The residue was treated with methyl iodide (0.5 mL, 8.1 mmol) and stirred overnight before being concentrated and purified by reverse phase HPLC to provide 1-{[3-(1-(6-chloro-3′-ethyl-2-biphenylyl)-1-hydroxy-4-{[(methyloxy)carbonyl]amino}butyl)-1-piperidinyl]carbonyl}-3-methyl-1H-imidazol-3-ium. MS (m/z) 553.2 (M⁺)

Step 2. methyl {(4S)-4-(6-chloro-3′-ethyl-2-biphenylyl)-4-hydroxy-4-[(3R)-1-(1-piperazinylcarbonyl)-3-piperidinyl]butyl}carbamate: A solution of 1-{[3-(1-(6-chloro-3′-ethyl-2-biphenylyl)-1-hydroxy-4-{[(methyloxy)carbonyl]amino}butyl)-1-piperidinyl]carbonyl}-3-methyl-1H-imidazol-3-ium (0.08 g, 0.14 mmol) in 1 mL of CH₃CN was treated with tert-butyl 1-piperazinecarboxylate (0.05 g, 0.28 mmol) and heated at 50° C. overnight before being subjected to reverse phase HPLC and concentration under reduced pressure. The residue was dissolved in 1.5 mL of CH₃CN, treated with 1.5 mL of 2N aqueous HCl, and stirred at 25 C overnight. The mixture was concentrated under reduced pressure to provide methyl {4-(6-chloro-3′-ethyl-2-biphenylyl)-4-hydroxy-4-[(3R)-1-(1-piperazinylcarbonyl)-3-piperidinyl]butyl}carbamate as a white solid. MS (m/z) 557.3 (M+H⁺).

### Example 5

The following piperidines were prepared following procedures analogous to those described in Example 4:
#28 methyl (4-(6-chloro-3′-ethyl-2-biphenylyl)-4-hydroxy-4-{(3R)-1-[(4-piperidinylamino)carbonyl]-3-piperidinyl}butyl)carbamate using 1,1-dimethylethyl 4-amino-1-piperidinecarboxylate instead of tert-butyl 1-piperazinecarboxylate in Step 2.
#29 methyl [4-(6-chloro-3′-ethyl-2-biphenylyl)-4-hydroxy-4-((3R)-1-{[(4-piperidinylmethyl)amino]carbonyl}-3-piperidinyl)butyl]carbamate using 1,1-dimethylethyl 4-(aminomethyl)-1-piperidinecarboxylate instead of 1,1-dimethylethyl 4-amino-1-piperidinecarboxylate instead of tert-butyl 1-piperazinecarboxylate in Step 2.

### Example 6

### methyl {4-(3′-ethyl-6-fluoro-2-biphenylyl)-4-hydroxy-4-[(3R)-1-(2-morpholinylacetyl)-3-piperidinyl]butyl}carbamate (#21)

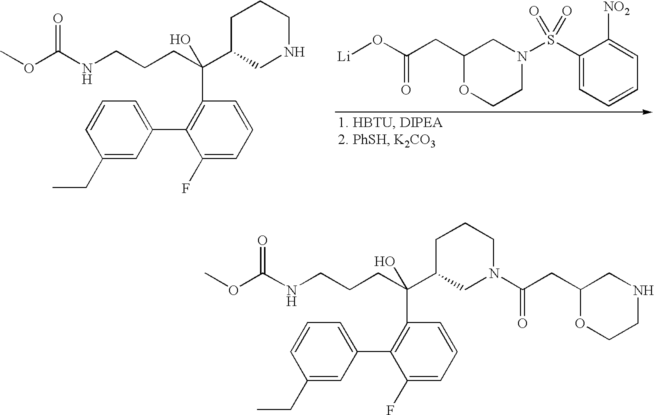

### Step 1. Methyl {4-(3′-ethyl-6-fluoro-2-biphenylyl)-4-hydroxy-4-[(3R)-1-({4-[(2-nitrophenyl)sulfonyl]-2-morpholinyl}acetyl)-3-piperidinyl]butyl}carbamate

A solution of methyl {4-(3′-ethyl-6-fluoro-2-biphenylyl)-4-hydroxy-4-[(3R)-3-piperidinyl]butyl}carbamate (0.080 g, 0.17 mmol) and lithium {4-[(2-nitrophenyl)sulfonyl]-2-morpholinyl}acetate (˜0.25 mmol) in 1.5 mL of DMF at 25° C. was treated with i-Pr₂EtN (0.050 mL, 0.29 mmol) and HBTU (0.070 g, 0.18 mmol) and the mixture was stirred for 3 hours before being concentrated under reduced pressure and subjected to reverse phase HPLC to give methyl {4-(3′-ethyl-6-fluoro-2-biphenylyl)-4-hydroxy-4-[(3R)-1-({4-[(2-nitrophenyl)sulfonyl]-2-morpholinyl}acetyl)-3-piperidinyl]butyl}carbamate as an oil (0.041 g, 34%). ESI-MS (m/z): 741.3 (M+H⁺).

### Step 2. Methyl {4-(3′-ethyl-6-fluoro-2-biphenylyl)-4-hydroxy-4-[(3R)-1-(2-morpholinylacetyl)-3-piperidinyl]butyl}carbamate

A solution of methyl {4-(3′-ethyl-6-fluoro-2-biphenylyl)-4-hydroxy-4-[(3R)-1-({4-[(2-nitrophenyl)sulfonyl]-2-morpholinyl}acetyl)-3-piperidinyl]butyl}carbamate (40 mg, 0.054 mmol) in DMF (1 mL) at 25° C. was treated with K₂CO₃ (15 mg, 0.11 mmol) and benzenethiol (0.016 mL, 0.16 mmol) and the mixture was stirred overnight before being filtered and subjected to reverse phase HPLC to provide methyl {4-(3′-ethyl-6-fluoro-2-biphenylyl)-4-hydroxy-4-[(3R)-1-(2-morpholinylacetyl)-3-piperidinyl]butyl}carbamate as an oil (29 mg, 95%). ESI-MS (m/z): 556.3 (M+H⁺).

The following are compounds of the invention:

| | | Synthetic | | | |
|---|---|---|---|---|---|
| | | Method | LC_MS | | Mass |
| Cpd. #. | Name | Example No. | Method | t_{R} (min) | observed |
| 1 | methyl 4-(3′-ethyl-6-fluorobiphenyl-2-yl)-4-hydroxy- | 3 | 1 | 2.28 | 542.3 |
| | 4-((R)-1-((2S,4R)-4-hydroxypyrrolidine-2- | | | | |
| | carbonyl)piperidin-3-yl)butylcarbamate | | | | |
| 2 | methyl (4-(3′-ethyl-6-fluoro-2-biphenylyl)-4- | 3 | 1 | 2.43 | 526.3 |
| | hydroxy-4-{(3R)-1-[(3R)-3-pyrrolidinylcarbonyl]-3- | | | | |
| | piperidinyl}butyl)carbamate | | | | |
| 3 | ((3R)-3-(1-hydroxy-5-methoxy-1-(2- | 1 | | | |
| | phenoxyphenyl)pentyl)piperidin-1-yl)(piperidin-4- | | | | |
| | yl)methanone | | | | |
| 4 | methyl (4-(3′-ethyl-6-fluoro-2-biphenylyl)-4- | 3 | 1 | 2.37 | 540.3 |
| | hydroxy-4-{(3R)-1-[(2S)-2-pyrrolidinylacetyl]-3- | | | | |
| | piperidinyl}butyl)carbamate | | | | |
| 5 | methyl {4-(3′-ethyl-6-fluoro-2-biphenylyl)-4- | 3 | 1 | 2.35 | 540.3 |
| | hydroxy-4-[(3R)-1-(2-piperidinylcarbonyl)-3- | | | | |
| | piperidinyl]butyl}carbamate | | | | |
| 6 | methyl (4-(3′-ethyl-6-fluoro-2-biphenylyl)-4- | 3 | 1 | 2.33 | 540.3 |
| | hydroxy-4-{(3R)-1-[(3R)-3-piperidinylcarbonyl]-3- | | | | |
| | piperidinyl}butyl)carbamate | | | | |
| 7 | methyl {4-(3′-ethyl-6-fluoro-2-biphenylyl)-4- | 3 | 1 | 2.29 | 540.3 |
| | hydroxy-4-[(3R)-1-(4-piperidinylcarbonyl)-3- | | | | |
| | piperidinyl]butyl}carbamate | | | | |
| 8 | methyl {4-(3′-ethyl-6-fluoro-2-biphenylyl)-4- | 3 | 1 | 2.19 | 554.3 |
| | hydroxy-4-[(3R)-1-(4-piperidinylacetyl)-3- | | | | |
| | piperidinyl]butyl}carbamate | | | | |
| 9 | methyl {4-(3′-ethyl-6-fluoro-2-biphenylyl)-4- | 3 | 1 | 2.47 | 554.3 |
| | hydroxy-4-[(3R)-1-(3-piperidinylacetyl)-3- | | | | |
| | piperidinyl]butyl}carbamate | | | | |
| 10 | methyl (4-(3′-ethyl-6-fluoro-2-biphenylyl)-4- | 3 | 1 | 2.43 | 568.3 |
| | hydroxy-4-{(3R)-1-[3-(4-piperidinyl)propanoyl]-3- | | | | |
| | piperidinyl}butyl)carbamate | | | | |
| 11 | methyl (4-(3′-ethyl-6-fluoro-2-biphenylyl)-4- | 3 | 1 | 2.39 | 568.3 |
| | hydroxy-4-{(3R)-1-[3-(2-piperidinyl)propanoyl]-3- | | | | |
| | piperidinyl}butyl)carbamate | | | | |
| 12 | methyl (4-(3′-ethyl-6-fluoro-2-biphenylyl)-4-{(3R)-1- | 3 | 1 | 2.28 | 597.3 |
| | [(1-glycyl-4-piperidinyl)carbonyl]-3-piperidinyl}-4- | | | | |
| | hydroxybutyl)carbamate | | | | |
| 13 | methyl (4-(3′-ethyl-6-fluoro-2-biphenylyl)-4- | 3 | 1 | 2.41 | 609.3 |
| | hydroxy-4-{(3R)-1-[1-(4-piperidinyl)-L-prolyl]-3- | | | | |
| | piperidinyl}butyl)carbamate | | | | |
| 14 | methyl {4-(3′-ethyl-6-fluoro-2-biphenylyl)-4- | 3 | 1 | 2.48 | 673.3 |
| | hydroxy-4-[(3R)-1-(L-phenylalanyl-L-prolyl)-3- | | | | |
| | piperidinyl]butyl}carbamate | | | | |
| 15 | methyl [4-(3′-ethyl-6-fluoro-2-biphenylyl)-4- | 3 | 1 | 2.44 | 599.3 |
| | hydroxy-4-((3R)-1-{[4-(2-hydroxyethyl)-1- | | | | |
| | piperazinyl]acetyl}-3-piperidinyl)butyl]carbamate | | | | |
| 16 | methyl {4-(6-chloro-3′-ethyl-2-biphenylyl)-4- | 4 | 1 | 2.49 | 557.3 |
| | hydroxy-4-[(3R)-1-(1-piperazinylcarbonyl)-3- | | | | |
| | piperidinyl]butyl}carbamate | | | | |
| 17 | methyl [4-(6-chloro-3′-methyl-2-biphenylyl)-4- | 3 | 1 | 2.73 | 571.2 |
| | ((3R)-1-{[(4S)-2,6-dioxohexahydro-4- | | | | |
| | pyrimidinyl]carbonyl}-3-piperidinyl)-4- | | | | |
| | hydroxybutyl]carbamate | | | | |
| 18 | methyl [4-(6-chloro-3′-methyl-2-biphenylyl)-4- | 3 | 1 | 2.54 | 571.2 |
| | ((3R)-1-{[(4R)-2,6-dioxohexahydro-4- | | | | |
| | pyrimidinyl]carbonyl}-3-piperidinyl)-4- | | | | |
| | hydroxybutyl]carbamate | | | | |
| 19 | methyl (4-(3′-ethyl-6-fluoro-2-biphenylyl)-4- | 3 | 1 | 2.49 | 616.3 |
| | hydroxy-4-{(3R)-1-[(4-phenyl-4- | | | | |
| | piperidinyl)carbonyl]-3-piperidinyl}butyl)carbamate | | | | |
| 20 | methyl {4-(3′-ethyl-6-fluoro-2-biphenylyl)-4- | 3 | 1 | 2.34 | 542.3 |
| | hydroxy-4-[(3R)-1-(2-morpholinylcarbonyl)-3- | | | | |
| | piperidinyl]butyl}carbamate | | | | |
| 21 | methyl {4-(3′-ethyl-6-fluoro-2-biphenylyl)-4- | 6 | 1 | 2.44 | 556.3 |
| | hydroxy-4-[(3R)-1-(2-morpholinylacetyl)-3- | | | | |
| | piperidinyl]butyl}carbamate | | | | |
| 22 | 1-(6-chloro-3′-ethyl-2-biphenylyl)-5-(methyloxy)-1- | 3 | 1 | 3.01 | 526.2 |
| | [(3R)-1-(1H-tetrazol-5-ylacetyl)-3-piperidinyl]-1- | | | | |
| | pentanol | | | | |
| 23 | methyl {4-(6-chloro-3′-ethyl-2-biphenylyl)-4- | 3 | 1 | 2.8 | 555.2 |
| | hydroxy-4-[(3R)-1-(1H-tetrazol-5-ylacetyl)-3- | | | | |
| | piperidinyl]butyl}carbamate | | | | |
| 24 | N-{4-(6-chloro-3′-methyl-2-biphenylyl)-4-hydroxy- | 3 | 1 | 2.56 | 525.2 |
| | 4-[(3R)-1-(1H-tetrazol-5-ylacetyl)-3- | | | | |
| | piperidinyl]butyl}acetamide | | | | |
| 25 | N-{4-(6-chloro-3′-methyl-2-biphenylyl)-4-hydroxy- | 3 | 1 | 2.41 | 523.2 |
| | 4-[(3R)-1-(1H-imidazol-4-ylacetyl)-3- | | | | |
| | piperidinyl]butyl}acetamide | | | | |
| 26 | methyl {4-(3′-ethyl-6-fluoro-2-biphenylyl)-4- | 3 | 1 | 2.48 | 534.2 |
| | hydroxy-4-[(3R)-1-(4-pyridinylcarbonyl)-3- | | | | |
| | piperidinyl]butyl}carbamate | | | | |
| 27 | methyl {4-(3′-ethyl-6-fluoro-2-biphenylyl)-4- | 3 | 1 | 2.45 | 548.3 |
| | hydroxy-4-[(3R)-1-(4-pyridinylacetyl)-3- | | | | |
| | piperidinyl]butyl}carbamate | | | | |
| 28 | methyl (4-(6-chloro-3′-ethyl-2-biphenylyl)-4- | 5 | 1 | 2.48 | 571.2 |
| | hydroxy-4-{(3R)-1-[(4-piperidinylamino)carbonyl]- | | | | |
| | 3-piperidinyl}butyl)carbamate | | | | |
| 29 | methyl [4-(6-chloro-3′-ethyl-2-biphenylyl)-4- | 5 | 1 | 2.41 | 585.2 |
| | hydroxy-4-((3R)-1-{[(4- | | | | |
| | piperidinylmethyl)amino]carbonyl}-3- | | | | |
| | piperidinyl)butyl]carbamate | | | | |
| 30 | methyl {4-(6-chloro-3′-ethyl-2-biphenylyl)-4- | 3 | 2 | 1.23 | 604.3 |
| | hydroxy-4-[(3R)-1-(1,2,3,4-tetrahydro-6- | | | | |
| | isoquinolinylcarbonyl)-3- | | | | |
| | piperidinyl]butyl}carbamate | | | | |
| 31 | methyl {4-(6-chloro-3′-ethyl-2-biphenylyl)-4- | 3 | 2 | 1.24 | 604.3 |
| | hydroxy-4-[(3R)-1-(1,2,3,4-tetrahydro-7- | | | | |
| | isoquinolinylcarbonyl)-3- | | | | |
| | piperidinyl]butyl}carbamate | | | | |

### Example 7

### In Vitro Activity Studies

The compounds of the invention have enzyme-inhibiting properties. In particular, they inhibit the action of the natural enzyme renin. The latter passes from the kidneys into the blood where it affects the cleavage of angiotensinogen, releasing the decapeptide angiotensin I which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors in vitro is demonstrated experimentally by means of a test which measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increase as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

In this in vitro system the compounds of the invention exhibit 50% inhibition at concentrations of from approximately 5000 nM to approximately 0.01 nM. Preferred compounds of the invention exhibit 50% inhibition at concentrations of from approximately 50 nM to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 8

### In Vitro Activity Studies

All reactions are carried out in a low volume, black, 384 well microtiter plate (greiner bio-one). Compounds were diluted in 100% DMSO, and a 100 nL aliquot of each compound concentration was stamped into the plate using a Hummingbird (Genomic Solutions). 5 μL of 600 pM renin (trypsin-activated recombinant human renin) was then added to the plate, followed by 5 μL of 2 μM substrate (Arg-Glu-Lys(5-FAM)-Ile-His-Pro-Phe-His-Leu-Val-Ile-His-Thr-Lys(5,6-TAMRA)-Arg-CONH₂). Both renin and substrate were made up in buffer containing 50 mM HEPES, 125 mM NaCl, 0.1% CHAPS, with the pH adjusted to 7.4. After 2 hours of reaction at room temperature, the plates were read on a Viewlux (PerkinElmer) with an excitation/emission of 485/530 nm, and using a 505 nm cutoff filter. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. In the in vitro systems described above, the compounds of the invention exhibit an IC₅₀ for renin of between about 5,000 nM to about 0.01 nM; preferred compounds exhibit an IC₅₀ for renin of between about 50 nM to about 0.01 nM; and more preferred compounds exhibit an IC₅₀ for renin of between about 5 nM to about 0.01 nM.

### Example 9

### In Vitro Activity Studies

The potency of renin inhibitors was measured using an in vitro renin assay. In this assay, renin-catalyzed proteolysis of a fluorescently labeled peptide converts the peptide from a weakly fluorescent to a strongly fluorescent molecule. The following test protocol was used. Substrate solution (5 μl; 2 μM Arg-Glu-Lys(5-Fam)-Ile-His-Pro-Phe-His-Leu-Val-Ile-His-Thr-Lys(5,6 Tamra)-Arg-CONH₂ in 50 mM Hepes, 125 mM NaCl, 0.1% CHAPS, pH 7.4) then trypsin-activated recombinant human renin (Scott, Martin J. et. al. Protein Expression and Purification 2007, 52(1), 104-116; 5 μL; 600 pM renin in 50 mM Hepes, 125 mM NaCl, 0.1% CHAPS, pH 7.4) were added sequentially to a black Greiner low volume 384-well plate (cat. #784076) pre-stamped with a 100 nl DMSO solution of compound at the desired concentration. The assay plates were incubated at room temperature for 2 hours with a cover plate then quenched by the addition of a stop solution (2 μL; 5 μM of Bachem C-3195 in 50 mM Hepes, 125 mM NaCl, 0.1% CHAPS, pH 7.4, 10% DMSO). The assay plates were read on an LJL Acquest using a 485 nm excitation filter, a 530 nm emission filter, and a 505 nm dichroic filter. Compounds were initially prepared in neat DMSO at a concentration of 10 mM. For inhibition curves, compounds were diluted using a three fold serial dilution and tested at 11 concentrations (e.g. 50 μM-0.8 nM or 25 μM-0.42 nM or 2.5 μM to 42 pM). Curves were analyzed using ActivityBase and XLfit, and results were expressed as pIC₅₀ values.

### Example 10

### In Vitro Activity of the Disclosed Compounds in Human Plasma

The action of renin inhibitors in vitro in human plasma is demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin IS added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are used for deriving percent of inhibition for each concentration of inhibitors and fitting dose-response data into a four parametric model from which IC₅₀ values, defined as concentrations of inhibitors at which 50% inhibition occurs, is determined.

### Example 11

### Efficacy of the Disclosed Inhibitors in a Transgenic Rat Model

The efficacy of the renin inhibitors is also evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments are conducted in 5-10 week-old double transgenic rats (dTGRs). The model has been described in detail earlier. Briefly, the human renin construct are used to generate transgenic animals (hRen) made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences are used to generate rats producing human angiotensinogen (hAogen). The hRen and hAogen rats are rederived using embryo transfer from breeding pairs obtained under license from Ascencion Gmbh (Germany). The hAogen and hRen are then crossed to produce the double transgenic dTGR) off-spring. The dTGr rats are maintained on irradiated rodent chow (5VO2, Purina Mills Inc) and normal water. Radio telemetry transmitters (TA11PAC40, Data Sciences International) are surgically implanted at 5-6 weeks of age. The telemetry system provided 24-h recordings of systolic, mean, diastolic arterial pressure (SAP, MAP, DAP, respectively) and heart rate (HR). Prior to dosing, baseline hemodynamic measures are obtained for 24 hours. Rats are then dosed orally with vehicle or drug and monitored up to 48 hours post-dose.

While this invention has been particularly shown and described with references to preferred embodiments thereof, it will be understood by those skilled in the art that various changes in form and details may be made therein without departing from the scope of the invention encompassed by the appended claims.

1. A compound having Formula I: 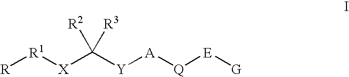
wherein:
R is:
a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;
b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or
c) a divalent radical selected from —(CH₂)₃—, —(CH₂)₄—, —(CH₂)₅— and —(CH₂)₆—, which is attached to R¹ to form a fused or spiro-fused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl;
X and Y are each independently CH₂ or a single bond;
R² is a) —H; or b) (C₂-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkanesulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:
1) 1 to 5 halogen atoms; and/or
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
wherein the divalent sulfur atoms in R² are independently optionally oxidized to sulfoxide or sulfone and wherein the carbonyl groups are optionally independently changed to a thiocarbonyl groups;
R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that
i) R² and R³ are not both hydrogen; and
ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:
1) 1 to 5 halogen atoms; and/or
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
the divalent sulfur atoms in R³ are independently optionally oxidized to sulfoxide or sulfone and wherein the carbonyl groups in R³ are optionally independently changed to thiocarbonyl groups;
A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups and oxo groups such that when there is substitution with one oxo group on a carbon atom it forms a carbonyl group and when there is substitution of one or two oxo groups on sulfur it forms sulfoxide or sulfone groups, respectively, where m is 1 to 3;
Q and Y are attached to carbon or nitrogen atoms in ring A in a 1,2 or 1,3, or 1,4 relationship;
Q is a divalent radical: 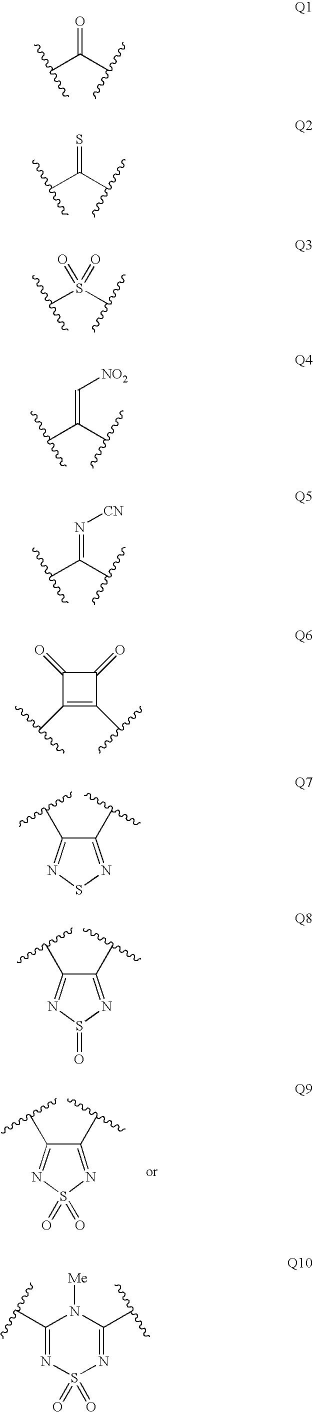 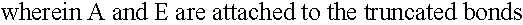
E is E¹ or —(C₁-C₃)alkyl-E¹; where E¹ is a 5-membered heteroaryl group containing 1-4 nitrogen atoms; an arylheterocyclyl group, wherein the heterocyclyl moiety contains 1-2 nitrogen atoms; or a saturated 4-, 5-, 6-, or 7-membered heterocyclic ring which is optionally bridged by (CH₂)ₙ via bonds to two members of the ring, wherein the ring is composed of carbon atoms and 1-3 heteroatoms selected from 1, 2, or 3 nitrogen atoms, 0 or 1 oxygen atoms, and 0 or 1 sulfur atoms; wherein E¹ is optionally substituted with one to three groups independently selected from halogen, hydroxy, (C₁-C₆)alkyl, halo(C₁-C₆)alkyl, hydroxy(C₁-C₆)alkyl, aryl, and oxo groups such that when there is substitution with one oxo group on a carbon atom it forms a carbonyl group and when there is substitution of one or two oxo groups on sulfur it forms sulfoxide or sulfone groups, respectively, where n is 1 to 3, and wherein G is attached to E¹ via a ring nitrogen atom, in a bonding arrangement illustrated as follows: 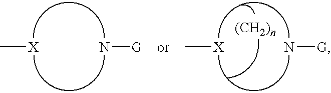
wherein X is a ring carbon atom or nitrogen atom bonded directly to Q or X is bonded to Q via the —(C₁-C₃)alkyl moiety of E;
and G is hydrogen, (C₁-C₆)alkyl, heterocyclyl, —(C₂-C₆)alkyl-OH, —(C₂-C₆)alkyl-NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino, where R^{4a} is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 nitrogen atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group;
or E¹ is a 6-membered heteroaryl group containing 1-2 nitrogen atoms which is optionally substituted with one to two groups independently selected from halogen, (C₁-C₆)alkyl, halo(C₁-C₆)alkyl, hydroxy(C₁-C₆)alkyl, and aryl, and G is absent;
or a salt thereof.

2. The compound or salt according to claim 1, wherein the compound of Formula I is represented by the following structural formula: 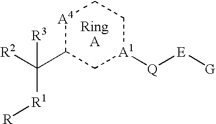
wherein when Ring A is a benzene ring, A¹ is C and A⁴ is CH and the bonds in ring A are aromatic bonds; when Ring A is a piperidinyl ring, A¹ is N, A⁴ is CH₂ and the bonds in ring A are single bonds; and when Ring A is a morpholinyl ring, A¹ is N, A⁴ is O and the bonds in ring A are single bonds.

3. The compound or salt according to claim 2, wherein the compound of Formula I is represented by the following structural formula:

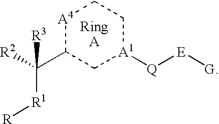

4. The compound or salt according to claim 2, wherein the compound of Formula I is represented by the following structural formula:

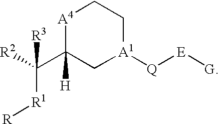

5. The compound or salt according to claim 1, wherein: E is E¹ or —(C₁-C₂)alkyl-E¹; where E¹ is a 5-membered heteroaryl group containing 1-4 nitrogen atoms; a 10-membered arylheterocyclyl group, wherein the heterocyclyl moiety contains 1-2 nitrogen atoms; or a saturated 5- or 6-membered heterocyclic ring, wherein the ring is composed of carbon atoms and 1 or 2 heteroatoms selected from 1 or 2 nitrogen atoms, 0 or 1 oxygen atoms, and 0 or 1 sulfur atoms, wherein E¹ is optionally substituted with 1-2 groups independently selected from halogen, hydroxy, (C₁-C₆)alkyl, halo(C₁-C₆)alkyl, hydroxy(C₁-C₆)alkyl, aryl, and oxo groups such that when there is substitution with one oxo group on a carbon atom it forms a carbonyl group and when there is substitution of one or two oxo groups on sulfur it forms sulfoxide or sulfone groups, respectively, wherein G is attached to ring E¹ via a ring nitrogen atom: 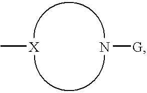
wherein X is a ring carbon atom or nitrogen atom bonded directly to Q X is bonded to Q via the —(C₁-C₂)alkyl moiety of E;
or E¹ is a 6-membered heteroaryl group containing 1-2 nitrogen atoms which is optionally substituted with one to two groups independently selected from halogen, hydroxy, (C₁-C₆)alkyl, halo(C₁-C₆)alkyl, hydroxy(C₁-C₆)alkyl, and aryl; and G is absent.

6. The compound or salt according to claim 1, wherein: E is E¹ or —(C₁-C₂)alkyl-E¹; where E¹ is a 5-membered heteroaryl group containing 1-4 nitrogen atoms; a 10-membered arylheterocyclyl group, wherein the heterocyclyl moiety contains one nitrogen atom; or a saturated 5- or 6-membered heterocyclic ring, wherein said ring is composed of carbon atoms and 1 or 2 heteroatoms selected from 1 or 2 nitrogen atoms and 0 or 1 oxygen atoms, wherein E¹ is optionally substituted with 1-2 groups independently selected from hydroxy, (C₁-C₄)alkyl, halo(C₁-C₄)alkyl, hydroxy(C₁-C₄)alkyl, aryl, and oxo groups;
or E¹ is a 6-membered heteroaryl group containing one nitrogen atom which is optionally substituted with one group selected from (C₁-C₄)alkyl, halo(C₁-C₄)alkyl, and hydroxy(C₁-C₄)alkyl, and G is absent.

7. The compound or salt according to claim 1, wherein: E is E¹ or —(C₁-C₂)alkyl-E¹; where E¹ is selected from the group consisting of
a) piperidinyl, piperazinyl, and pyrrolidinyl, said group being optionally substituted with a hydroxy, (C₁-C₃)alkyl or halo(C₁-C₃)alkyl group; and
b) morpholinyl, tetrazolyl, imidazolyl, pyridinyl, and tetrahydroisoquinolinyl, said group being optionally substituted with a hydroxy, phenyl, (C₁-C₃)alkyl, or halo(C₁-C₃)alkyl group.

8. The compound or salt according to claim 1, wherein: A or Ring A is a piperidinyl ring or a morpholinyl ring.

9. The compound or salt according to claim 1, wherein: Q is Q1, Q2, Q3, or Q6.

10. The compound or salt according to claim 1, wherein: Q is Q1.

11. The compound or salt according to claim 1, wherein: G is hydrogen, heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₇)cycloalkyl, or —(C₁-C₄)alkyl(C₄-C₇)heterocyclyl, wherein the (C₁-C₄)alkyl, moiety of said —C(═O)(C₁-C₄)alkylaryl, —C(═O)(C₁-C₄)alkyl(C₄-C₇)heterocyclyl, —(C₁-C₄)alkyl(C₃-C₇)cycloalkyl and —(C₁-C₄)alkyl(C₄-C₇)heterocyclyl, is optionally substituted by amino, hydroxy, or (C₁-C₃)alkylamino, where R⁴ is H or (C₁-C₃)alkyl and R^{4a} is selected from H, (C₁-C₃)alkyl, heterocyclyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 2 nitrogen atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group.

12. The compound or salt according to claim 1, wherein: G is hydrogen, (C₄-C₆)heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylphenyl, —C(═O)(C₁-C₄)alkyl(C₄-C₆)heterocyclyl, —(C₁-C₄)alkyl(C₃-C₆)cycloalkyl, or —(C₁-C₄)alkyl(C₄-C₆)heterocyclyl, wherein the (C₁-C₄)alkyl moiety of said —C(═O)(C₁-C₄)alkylphenyl, —C(═O)(C₁-C₄)alkyl(C₄-C₆)heterocyclyl, —(C₁-C₄)alkyl(C₃-C₆)cycloalkyl and —(C₁-C₄)alkyl(C₄-C₆)heterocyclyl is optionally substituted by amino, hydroxy, or (C₁-C₃)alkylamino, where R⁴ is H or (C₁-C₃)alkyl and R^{4a} is selected from H, (C₁-C₃)alkyl, (C₃-C₆)cycloalkyl(C₁-C₄)alkyl, and (C₄-C₆)heterocyclyl(C₁-C₄)alkyl.

13. The compound or salt according to claim 1, wherein: G is hydrogen, (C₅-C₆)heterocyclyl, —(C₂-C₃)alkyl-OH, —C(═O)(C₁-C₂)alkyl-NR⁴R^{4a}, or —C(═O)(C₁-C₃)alkylphenyl, wherein the (C₁-C₃)alkyl moiety of said —C(═O)(C₁-C₃)alkylphenyl is substituted by amino or (C₁-C₃)alkylamino, where R⁴ is H or (C₁-C₃)alkyl and R^{4a} is H.

14. The compound or salt according to claim 1, wherein:
R is phenyl, naphthyl, monocyclic heteroaryl, bicyclic heteroaryl, phenoxy, monocyclic heteroaryloxy, phenyl(C₁-C₃)alkoxy, and monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio, and H₂NCO; or a divalent radical selected from —(CH₂)₄— and —(CH₂)₅—;
R¹ is a phenyl or a monocyclic heteroaryl ring, optionally substituted with up to four substituents independently selected from: halogen, cyano, (C₁-C₃)alkyl, halo(C₁-C₃)alkyl, (C₁-C₃)alkoxy, halo(C₁-C₃)alkoxy, and H₂NCO;
R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy;
R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy;
E is E¹ or —(C₁-C₂)alkyl-E¹; where E¹ is a saturated 5- or 6-membered heterocyclic ring, wherein said ring is composed of carbon atoms and 1 or 2 nitrogen atoms, said ring being optionally substituted with one group selected from hydroxy, (C₁-C₄)alkyl, halo(C₁-C₄)alkyl, and hydroxy(C₁-C₄)alkyl; or
E¹ is a 5-membered heteroaryl group containing 1-4 nitrogen atoms; a 10-membered arylheterocyclyl group, wherein the heterocyclyl moiety contains one nitrogen atom, wherein E¹ is optionally substituted with 1-2 groups independently selected from hydroxy, (C₁-C₄)alkyl, halo(C₁-C₄)alkyl, hydroxy(C₁-C₄)alkyl, aryl, and oxo groups; and
G is hydrogen, (C₅-C₆)heterocyclyl, —(C₂-C₃)alkyl-OH, —C(═O)(C₁-C₂)alkyl-NR⁴R^{4a}, or —C(═O)(C₁-C₃)alkylphenyl, wherein the (C₁-C₃)alkyl moiety of said —C(═O)(C₁-C₃)alkylphenyl is substituted by amino or (C₁-C₃)alkylamino, where R⁴ is H or (C₁-C₃)alkyl and R^{4a} is H, or a salt thereof;
or E¹ is a 6-membered heteroaryl group containing one nitrogen atom which is optionally substituted with one group selected from (C₁-C₄)alkyl, halo(C₁-C₄)alkyl, and hydroxy(C₁-C₄)alkyl, and G is absent;
A or Ring A is a piperidinyl ring or a morpholinyl ring; and Q is Q1.

15. The compound or salt according to claim 1, wherein:
R is phenyl, naphthyl, monocyclic heteroaryl, bicyclic heteroaryl, phenoxy, monocyclic heteroaryloxy, phenyl(C₁-C₃)alkoxy, and monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio, and H₂NCO; or a divalent radical selected from —(CH₂)₄— and —(CH₂)₅—;
R¹ is a phenyl or a monocyclic heteroaryl ring, optionally substituted with up to four substituents independently selected from: halogen, cyano, (C₁-C₃)alkyl, halo(C₁-C₃)alkyl, (C₁-C₃)alkoxy, halo(C₁-C₃)alkoxy, and H₂NCO;
R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy;
R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy;
A or Ring A is a piperidinyl ring or a morpholinyl ring;
Q is Q1;
E is E¹ or —(C₁-C₂)alkyl-E¹; where E¹ is a saturated 5- or 6-membered heterocyclic ring, wherein said ring is composed of carbon atoms and 1 or 2 nitrogen atoms, said ring being optionally substituted with one group selected from hydroxy, (C₁-C₄)alkyl, halo(C₁-C₄)alkyl, and hydroxy(C₁-C₄)alkyl;
G is hydrogen, (C₅-C₆)heterocyclyl, —(C₂-C₃)alkyl-OH, —C(═O)(C₁-C₂)alkyl-NR⁴R^{4a}, or —C(═O)(C₁-C₃)alkylphenyl, wherein the (C₁-C₃)alkyl moiety of said —C(═O)(C₁-C₃)alkyl-NR⁴R^{4a}, or substituted by amino or (C₁-C₃)alkylamino, where R⁴ is H or (C₁-C₃)alkyl and R^{4a} is H.

16. The compound or salt according to claim 1, wherein R is 3-methylphenyl, 3-ethylphenyl, or phenoxy; R¹ is 6-fluorophenyl, 6-chlorophenyl or phenyl; R² is 3-(N-acetylamino)propyl, 3-(methoxycarbonylamino)propyl, or 4-methoxybutyl; R³ is hydroxyl; A or Ring A is a piperidine ring; Q is Q1; E is piperidin-2-yl, piperidin-3-yl, piperidin-4-yl, 4-phenyl-piperidin-4-yl, piperazin-1-yl, morpholin-2-yl, pyrrolidin-2-yl, pyrrolidin-3-yl, 4-hydroxy-pyrrolidin-2-yl, piperidin-3-ylmethyl-, piperidin-4-ylmethyl-, piperazin-1-ylmethyl-, morpholin-2-ylmethyl-, pyrrolidine-2-ylmethyl-, piperidin-2-ylethyl-, piperidin-2-ylethyl-, piperidin-4-ylethyl-, 1H-tetrazol-5-ylmethyl-, 1H-imidazol-4-ylmethyl-, pyridine-4-yl, pyridine-4-ylmethyl-, tetrahydroisoquinolin-6-yl, and tetrahydroisoquinolin-7-yl; G is H, 2-hydroxyethyl-, aminoacetyl-, piperidin-4-yl, or (2-amino-3-phenyl)propanoyl-.

17. The compound or salt according to claim 1, wherein, R is 3-ethylphenyl or phenoxy; R¹ is 6-fluorophenyl, 6-chlorophenyl or phenyl; R² is 3-(methoxycarbonylamino)propyl or 4-methoxybutyl; R³ is hydroxyl; A or Ring A is a piperidine ring; Q is Q1; E is piperidin-2-yl, piperidin-3-yl, piperidin-4-yl, piperazin-1-yl, pyrrolidi-2-nyl, pyrrolidine-3-yl, 4-hydroxy-pyrrolidin-2-yl-, piperidin-3-ylmethyl-, piperidin-4-ylmethyl-, piperazin-1-ylmethyl-, pyrrolidine-2-ylmethyl-, piperidin-2-ylethyl-, piperidin-4-ylethyl-; G is H, 2-hydroxyethyl, aminoacetyl-, piperidin-4-yl, or (3-phenyl, 2-amino)propanoyl-.

18. The compound or salt according to claim 1, which is selected from Compounds I-1-I-31.

19. A pharmaceutical composition comprising the compound or salt according to claim 1, and a pharmaceutically acceptable carrier therefore.

20. The pharmaceutical composition according to claim 19, further comprising an additional agent selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

21. A method of inhibiting an aspartic protease, wherein the aspartic protease is renin, in a subject in need thereof comprising administering to the subject a therapeutically effective amount of the compound or salt of claim 1.

22. A method for treating or ameliorating an aspartic protease mediated disorder in a subject in need thereof comprising administering to said subject a therapeutically effective amount of the compound or salt of claim 1.

23. The method of claim 22, wherein the aspartic protease at least one of β-secretase, plasmepsin and HIV protease.

24. A method for treating or ameliorating a renin mediated disorder in a subject in need thereof comprising administering to the subject an effective amount of the compound or salt of claim 1.

25. The method of claim 24, wherein the renin mediated disorder is hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, post-surgical hypertension, restenosis following angioplasty, raised intra-ocular pressure, glaucoma, abnormal vascular growth, hyperaldosteronism, anxiety states, or a cognitive disorder.

26. A method for the treatment of hypertension in a subject in need thereof comprising administering to the subject the compound or salt of claim 1 in combination therapy with one or more additional agents, wherein each of said additional agents is independently selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

27. The method of claim 26, wherein: the α-blocker is selected from the group consisting of doxazosin, prazosin, tamsulosin, and terazosin; the β-blocker is selected from the group consisting of atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, and carvedilol, or pharmaceutically acceptable salts thereof; the calcium channel blocker is selected from the group consisting of dihydropyridines (DHPs) and non-DHPs, wherein the DHPs are selected from the group consisting of amlodipine, felodipine, ryosidine, isradipine, lacidipine, nicardipine, nifedipine, nigulpidine, nimodiphine, nisoldipine, nitrendipine, and nivaldipine and their pharmaceutically acceptable salts and the non-DHPs are selected from the group consisting of flunarizine, prenylamine, diltiazem, fendiline, gallopamil, mibefradil, anipamil, tiapamil, and verampimil, or pharmaceutically acceptable salts thereof; the diuretic is a thiazide derivative selected from the group consisting of an amiloride, chlorothiazide, hydrochlorothiazide, methylchlorothiazide, and chlorothalidon; the ACE inhibitor is selected from the group consisting of alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril; the dual angiotensin converting enzyme-neutral endopeptidase inhibitor is selected from the group consisting of include omapatrilat, fasidotril, and fasidotrilat; the angiotensin-receptor blocker is selected from the group consisting of candesartan, eprosartan, irbesartan, losartan, olmesartan, tasosartan, telmisartan, and valsartan; the aldosterone synthase inhibitor is selected from the group consisting of anastrozole, fadrozole, and exemestane; the aldosterone-receptor antagonist is selected from the group consisting of spironolactone and eplerenone; and the endothelin antagonist is selected from the group consisting of bosentan, enrasentan, atrasentan, darusentan, sitaxentan, and tezosentan, or pharmaceutically acceptable salts thereof.

28. The method of claim 27, wherein the compound and the additional agents are administered by sequential administration or simultaneous administration.

29-32. (canceled)
